(19) United States
(12) Patent Application Publication
(10) Pub. No.: US 2006/0004087 A1
(43) Pub. Date: Jan. 5, 2006

(54) Tetracyclic compounds as estrogen ligands

(75) Inventors: Christopher P. Miller, Wayne, PA (US); Michael D. Collini, Clifton Heights, PA (US); Robert L. Morris, Wayne, PA (US); Robert R. Singhaus JR., Pottstown, PA (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/170,017

(22) Filed: Jun. 29, 2005

Related U.S. Application Data
(60) Provisional application No. 60/584,516, filed on Jul. 1, 2004.

(51) Int. Cl. A61K 31/353; A61K 31/343; C07D 493/04

(52) U.S. Cl. 514/453; 514/468; 549/383; 549/457

(57) ABSTRACT

This invention provides estrogen receptor modulators having the structure: 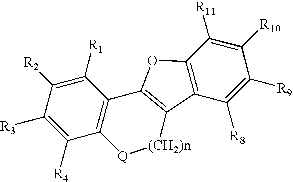
wherein R₁, R₂, R₃, R₄, Q, n, R₈, R₉, R₁₀, and R₁₁ have been defined in the specification; or a pharmaceutically acceptable salt thereof. The invention further provides methods for the preparation and use of the compounds.

### CROSS REFERENCE

This invention claims priority benefit of U.S. provisional application Ser. No. 60/584,516 filed Jul. 1, 2004, which is hereby incorporated by reference in its entirety.

### FIELD OF THE INVENTION

This invention relates to tetracyclic compounds which are useful as estrogenic agents, methods of preparing the compounds, and methods of using the compounds.

### BACKGROUND OF THE INVENTION

The pleiotropic effects of estrogens in mammalian tissues have been well documented. (Dey, M., Lyttle, C. R., Pickar, J. H. Maturitas (2000), 34(S2): S25-S33, Speroff, L., Ann. N.Y. Acad. Sci. (2000), 900, 26-39, Nozaki, M., Ernst Schering Res. Found. Workshop (2000), Suppl. 4,115-125). The estrogen receptor (ER), a member of the nuclear hormone ER family, regulates transcription through its interactions with a large number of proteins, including co-activators and co-repressors (collectively referred to as coregulators), and an estrogen response element (ERE). In addition to its ability to effect the cellular transcription machinery through the ERE, the ER also can affect transcriptional processes independent of its direct interaction with DNA. For example, it has been demonstrated that 17β-estradiol can inhibit IL-6 promoter activity. This inhibition requires 17β-estradiol binding to the ER, but does not depend on having a functional DNA-binding domain (Ray, A., Prefontaine, K. E., Ray, P. J., J. Biol. Chem. (1994), 269: 12940). Even the unliganded ER may affect the transcription process after phosphorylation of serine residues, especially in the AF-1 containing AB domains of the ER.

Recently, a second ER (ERβ) with high affinity for 17β-estradiol has been identified. A comparison of the physical structure of ERβ with the first to be identified ER (ERα) reveals that ERβ is shorter in length (530 AA vs. 595 AA), but contains the same functional domains. The AB domains of ERβ are somewhat truncated relative to ERα (148AA vs. 180AA) and not surprisingly, the AF-1 activation potential between the two ERs is different (McInerney, E. M., Weis, K. E., Sun, J., Mosselman, S., Katzenellenbogen, B. S., Endocrinology (1998), 139 (11): 4513-4522). The C domain (DNA-binding domain) displays remarkable homology between the two ERs (96%) and a fortiori, the two ERs would be expected to bind with similar affinities to a given ERE. However, although it has been shown that the two ERs bind to the EREs vitogenellin, c-fos, c-jun, pS2, cathepsin D, and acetylcholine transferase, they do not necessarily bind with the same affinity (Hyder, S. M., Chiappetta, C., Stancel, G. M., Biochem. Pharmacol. (1999) 57: 597-601). In contrast, the E domain (ligand binding domain or LBD) of the two ERs share only a 60% homology. However, structural analyses of the two ERs indicates that the residues in the ligand contact area are very similar, with only two residues different (ERα 421 (Met) ERβ 373(Ile); ERα 384 (Leu) ERα 336(Met)). Additionally, the variations in the overall sequence of the two ERs also may lead to different interactions between the subtypes and the various coregulatory proteins that enable or modify the ER transcriptional machinery. In fact, preliminary studies suggest that the coregulator SRC-3 interacts to a much greater extent with ERα than with ERβ. (Suen, C. S., Berrodin, T. J., Mastroeni, R., Cheskis, B. J., Lyttle, C. R., Frail, D., J. Biol. Chem. (1998), 273(42): 27645-27653).

Besides the differential interaction of the two ERs with various coregulatory proteins, the two ERs also have tissue distribution that is not coextensive. Even within a given tissue where both ERs are coexpressed there is sometimes localization of one of the ERs in a given cell-type. For example, in the human ovary, both ERα and ERβ RNA expression can be detected. Immunostaining demonstrates that ERβ is present in multiple cell types including granulosa cells in small, medium and large follicles, theca and corpora lutea, whereas ERα was weakly expressed in the nuclei of granulosa cells, but not in the theca nor in the corpora lutea (Taylor, A. H., Al-Azzawi, F., J. Mol. Endocrinol. (2000), 24(1): 145-155). In the endometrium, immunostaining showed both ERα and ERβ in luminal epithelial cells and in the nuclei of stromal cells, but significantly, ERβ appears to be weak or absent from endometrial glandular epithelia (Taylor, et al). Epithelial cells in most male tissues including the prostate, the urothelium and muscle layers of the bladder, and Sertoli cells in the testis, also are immunopositive for ERβ. Significant ERβ immunoreactivity has been detected in most areas of the brain, with the exception of the hippocampus, a tissue that stained positive for only ERα (ibid.).

Estrogens have been shown to exert a positive effect on the cardiovascular system that may help to explain the increased risk of cardiovascular disease observed in the post-menopause period. While some of the cardiovascular benefit may occur through estrogen action on the liver via upregulation of the LDL ER (thus, decreasing LDL levels, presumably an ER mediated response), it is also likely that direct action on the arterial wall has a role. It has been demonstrated that after a vascular injury event (denudation of rat artery), the ERβ message in the endothelial cells is upregulated by as much as 40 times that of ERα (Makela, S., Savolainen, H., Aavik, E., Myllarniemi, M., Strauss, L., Taskinen, E., Gustafsson, J. A., Hayry, P. (1999), 96(12): 7077-7082). In addition, 17β-estradiol was able to inhibit the vascular injury response in an ERα knockout mouse, although this same response also was inhibited in an ERβ knockout mouse (Lafrati, M. D., Karas, R. H., Aronovitz, M., Kim, S., Sullivan, Jr., T. R., Lubahn, D. B., O'Donnell, Jr., T. F., Korach, K. S., Mendelsohn, M. E., Nat. Med. (N.Y.) (1997), 3(5): 545-548; Karas, R. H., Hodgin, J. B., Kwoun, M., Krege, J. H., Aronovitz, M., Mackey, W., Gustafsson, J. A., Korach, K. S., Smithies, O., Mendelsohn, M. E., Proc. Natl. Acad. Sci. U.S.A. (1999), 96(26): 15133-15136). Provided that the response is not being inhibited by a yet unidentified ER, it is likely that the injury response could be inhibited by ligands that are selective for either one of the two ERs.

When the typical estrogen binds with an ER, the ER dissociates from HSP 90 as well as other molecular chaperones, and dimerizes with another ER. Since this mechanism of activation is shared by both ERs, the possibility exists for heterodimerization to take place in tissues where both ERs are expressed. Indeed, heterodimers of ERα and ERβ bind DNA with an affinity equal to that of ERα homodimers and greater than ERβ homodimers (Cowley, S. M., Hoare, S., Mosselman, S., Parker, M. G., J. Biol. Chem. (1997), 272(32): 19858-19862).

Despite the vast amount of work that has been done to date with respect to the effects of ER subtype signaling, clearly much still remains to be done. What is known is that treatment of patients with the classical estrogen agonists known to date, while often highly valuable and necessary to the patient, is not without its downside risks. Accordingly, there is a great unmet need in the art for novel estrogenic substances providing greater treatment options for the patient population. Subtype selective estrogens provide just such an alternative option and are provided for in the present invention.

### SUMMARY OF THE INVENTION

This invention provides compounds which possess demonstrable affinity for both ER α and ER β. The invention further provides processes for the preparation of the compounds, and uses therefor. In some embodiments, the compounds have the Formula I: 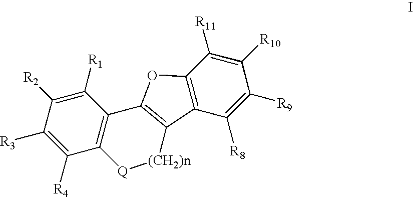
wherein:
Q has the structure II, III or IV: 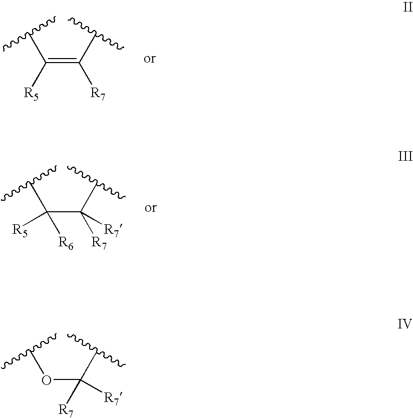
   R₁, R₄, R₅, R₆, R₇, R_{7′}, R₈ and R₁₁ are each independently selected from the group consisting of hydrogen, C₁-C₆ alkyl, —OR₂₀, halogen, —CF₃, —CF₂CF₃, —CH₂CF₃, —SR₂₀, NR₂OR₂₁, —CN, —CH₂CN, —CH₂CH₂CN, —CH═CHCN, —NO₂, —CH₂NO₂, —CH₂CH₂NO₂, —CH═CHNO₂ and —COR₂₀;
n=0 or 1;
   each R₂₀ and R₂₁ is independently selected from the group consisting of hydrogen, C₁-C₆ alkyl, —CF₃, benzyl, —CO₂(C₁-C₆alkyl) and —CO(C₁-C₆ alkyl);
      provided that:
   a) one of R₂ or R₃ must be —OR₂₀;
   b) one of R₉ or R₁₀ must be —OR₂₀;
   c) when R₂ is —OR₂₀, then R₁ and R₃ are independently selected from the group consisting of hydrogen, halogen, C₁-C₆ alkyl, —CF₃′—CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   d) when R₃ is —OR₂₀, then R₂ and R₄ are independently selected from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   e) when R₉ is —OR₂₀, then R₈ and R₁₀ are independently selected from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO_{2′}—CH₂CH₂NO₂, —CH═CHNO₂ and —COR₂₀;
   f) when R₁₀ is —OR₂₀, then R₉ and R₁₁ are independently selected from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀; and
   g) when Q has the structure IV, and R₇, R_{7′}, R₈, R₉, R₁₁ are each H, and n=0, then R₁₀ is not OR₂₀;
   or pharmaceutically acceptable salts thereof.

In some embodiments, Q has the structure II. In some such embodiments, R₃ and R₉ are each independently OR₂₀. In further such embodiments, R₃ and R₁₀ are each independently OR₂₀. In further such embodiments, R₂ and R₉ are each independently OR₂₀. In further such embodiments, R₂ and R₁₀ are each independently OR₂₀.

In some embodiments where Q has the structure II and R₃ and R₉ are each independently OR₂₀, R₁, R₂, R₄, R₈ and R₁₀ are each independently hydrogen or halogen; and R₁₁ is CN, halogen, methoxy, CH₂CN, NO₂ or C₁-C₆ alkyl. In some such embodiments, n is 0. In other such embodiments, n is 1.

In some embodiments, Q has the structure III. In some such embodiments, R₃ and R₉ are each independently OR₂₀. In further such embodiments, R₃ and R₁₀ are each independently OR₂₀. In further such embodiments, R₂ and R₉ are each independently OR₂₀. In further such embodiments, R₂ and R₁₀ are each independently OR₂₀.

In some embodiments where Q has the structure IV and R₃ and R₉ are each independently OR₂₀, R₂, R₄, R₈ and R₁₀ are each independently hydrogen or halogen; and R₁, is CN, halogen, methoxy, CH₂CN, NO₂ or C₁-C₆ alkyl. In some such embodiments, n is 0. In further such embodiments, n is 1.

In some embodiments, Q has the structure IV. In some such embodiments, R₃ and R₉ are each independently OR₂₀. In further such embodiments, R₃ and R₁₀ are each independently OR₂₀. In further such embodiments, R₂ and R₉ are each independently OR₂₀. In still further such embodiments, R₂ and R₁₀ are each independently OR₂₀.

In some embodiments where Q has the structure IV and R₃ and R₉ are each independently OR₂₀, R₂, R₄, R₈ and R₁₀ are each independently hydrogen or halogen; and R₁, is CN, halogen, methoxy, CH₂CN, NO₂ or C₁-C₆ alkyl. In some such embodiments, n is 0. In further such embodiments, n is 1.

The present invention further provides compounds having the structure: 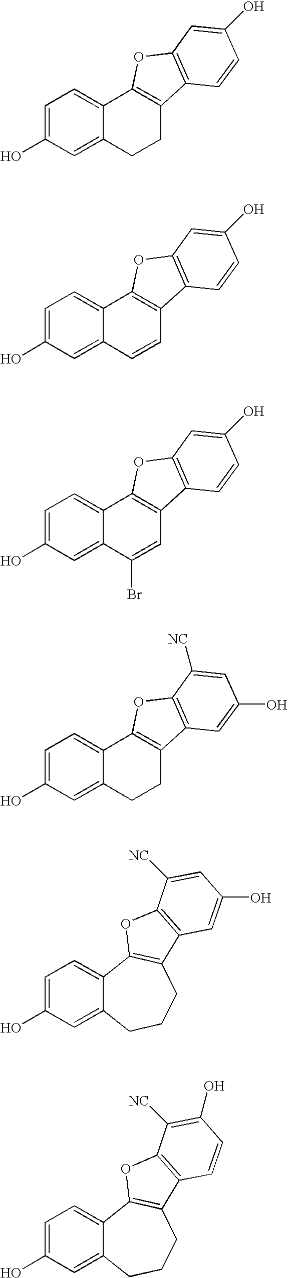 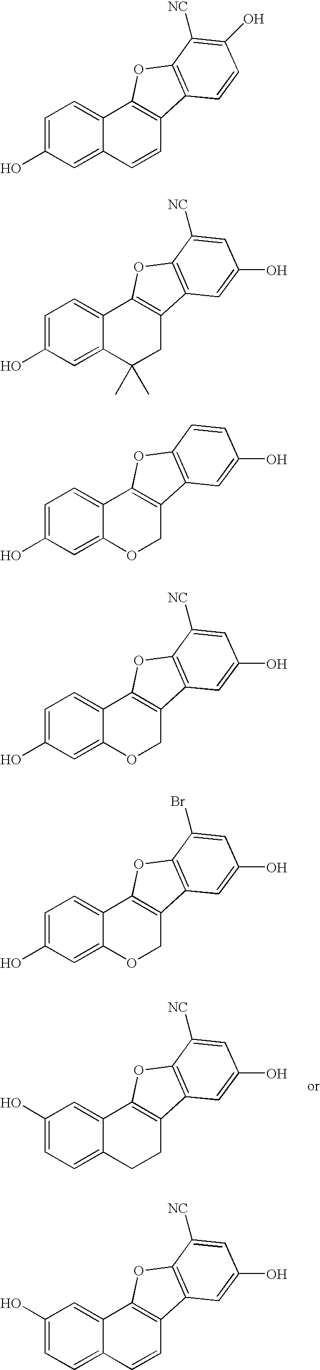
or pharmaceutically acceptable salts of each thereof.

In a further aspect, the invention provides methods of treating or inhibiting osteoporosis or inhibiting bone demineralization in a mammal, which comprises providing to said mammal an effective amount of a compound of the invention.

In a further aspect, the invention provides methods of treating or inhibiting inflammatory bowel disease, Crohn's disease, ulcerative proctitis, or colitis in a mammal, which comprises providing to said mammal an effective amount of a compound of the invention.

In a further aspect, the invention provides methods of treating or inhibiting prostatic hypertrophy, uterine leiomyomas, breast cancer, polycystic ovary syndrome, endometrial polyps, benign breast disease, adenomyosis, ovarian cancer, melanoma, prostate cancer, colon cancer, glioma or astioblastomia in a mammal, which comprises providing to said mammal an effective amount of a compound of the invention.

In a further aspect, the invention provides methods of lowering cholesterol, triglycerides, Lp(a), or LDL levels; inhibiting or treating hypercholesteremia, hyperlipidemia, cardiovascular disease, atherosclerosis, peripheral vascular disease, restenosis, or vasospasm; or inhibiting vascular damage in a mammal, which comprises providing to said mammal an effective amount of a compound of the invention.

In a further aspect, the invention provides methods of providing cognition enhancement or neuroprotection; or treating or inhibiting senile dementias, Alzheimer's disease, cognitive decline, stroke, anxiety, or neurodegenrative disorders in a mammal, which comprises providing to said mammal an effective amount of a compound of the invention.

In a further aspect, the invention provides methods of treating or inhibiting free radical induced disease states in a mammal, which comprises providing to said mammal an effective amount of a compound of the invention.

In a further aspect, the invention provides methods of treating or inhibiting vaginal or vulvar atrophy, atrophic vaginitis, vaginal dryness, pruritus, dyspareunia, dysuria, frequent urination, urinary incontinence, urinary tract infections in a mammal, which comprises providing to said mammal an effective amount of a compound of the invention.

In a further aspect, the invention provides methods of treating or inhibiting vasomotor symptoms in a mammal, which comprises providing to said mammal an effective amount of a compound of the invention.

In a further aspect, the invention provides methods of contraception in a mammal, which comprises providing to said mammal an effective amount of a compound of the invention.

In a further aspect, the invention provides methods of treating or inhibiting rheumatoid arthritis, osteoarthritis, or spondyloarthropathies in a mammal, which comprises providing to said mammal an effective amount of a compound of the invention.

In a further aspect, the invention provides methods of treating or inhibiting joint damage secondary to arthroscopic or surgical procedures in a mammal, which comprises providing to said mammal an effective amount of a compound of the invention.

In a further aspect, the invention provides methods of treating or inhibiting fertility in a mammal, which comprises providing to said mammal an effective amount of a compound of the invention.

In a further aspect, the invention provides methods of treating or inhibiting ischemia, reperfusion injury, asthma, pleurisy, multiple sclerosis, systemic lupus erythematosis, uveitis, sepsis, hemorrhagic shock, or type II diabetes in a mammal, which comprises providing to said mammal an effective amount of a compound of the invention.

Also provided in accordance with the present invention are pharmaceutical compositions comprising one or more compounds of the invention, and one or more pharmaceutically acceptable carriers. In some embodiments, the pharmaceutical composition includes one or more of 5,6-dihydro-benzo[b]naphtho[2,1-d]furan-3,9-diol, benzo[b]naphtho[2,1-d]furan-3,9-diol, 5-bromo-benzo[b]naphtho[2,1-d]furan-3,9-diol, 3,8-dihydroxy-5,6-dihydro-benzo[b]naphtho[2,1-d]furan-10-carbonitrile, 3,9-dihydroxy-6,7-dihydro-5H-12-oxa-dibenzo[a,e]azulen-11-carbonitrile, 3,9-dihydroxy-5,6-dihydro-benzo[b]naphtho[2,1-d]furan-10-carbonitrile, 3,9-dihydroxy-benzo[b]naphtho[2,1-d]furan-10-carbonitrile, 3,8-dihydroxy-5,5-dimethyl-5,6-dihydro-benzo[b]naphtho[2,1-d]furan-10-carbonitrile, 6H-benzo[4,5]furo[3,2-c]chromen-3,8-diol, 3,8-dihydroxy-6H-Benzo[4,5]furo[3,2-c]chromene-10-carbonitrile, 10-bromo-6H-benzo[4,5]furo[3,2-c]chromene-3,8-diol, 2,9-dihydroxy-5,6-dihydro-benzo[b]naphtho[2,1-d]furan-10-benzonitrile, 2,9-dihydroxy-benzo[b]naphtho[2,1-d]furan-10-carbonitrile, and one or more pharmaceutically acceptable carriers.

In a further aspect, the present invention provides processes for the preparation of a compound of the invention comprising the steps of:
a) coupling a compound of Formula V 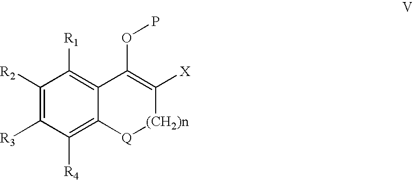
   wherein X is Cl, Br, or I; P is a protecting group; and the other constituent variables are as defined above;
   with a compound of Formula VI 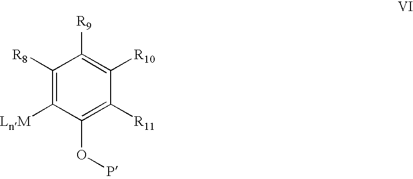
wherein:
M is a metal; L is a ligand; P′ is H or a protecting group; n′ is an integer from 0 to 5; and the other constituent variables are as defined above;
to form a compound of Formula VII; and 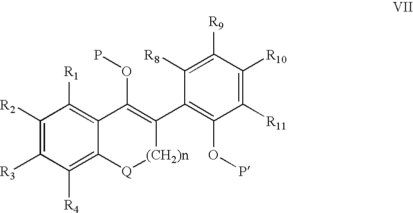
b) removing the groups P and P′ and cyclizing the resulting deprotected compound to form a compound of Formula I:

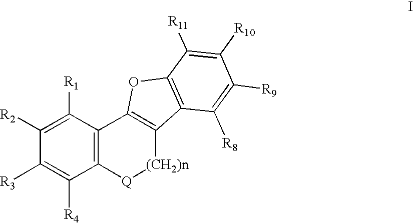

In some embodiments, P is Si(R′)₃, COC₁-C₆ alkyl, COOC₁-C₆ alkyl, CObenzyl, CO₂benzyl or C₁-C₆ alkyl; each R′ is independently C₁-C₆ alkyl or phenyl; and P′ is H, Si(R′)₃, COC₁-C₆ alkyl, COOC₁-C₆ alkyl, CObenzyl or C₁-C₆ alkyl; wherein each R′ is independently C₁-C₆ alkyl or phenyl.

In some such embodiments, P is COC₁-C₆ alkyl, COOC₁-C₆ alkyl, CObenzyl or CO₂benzyl; P′ is C₁-C₆ alkyl; and either a) M is B, L is (OH) or (OC₁-C₆ alkyl), and n′ is 2; or b) M is Sn, L is (C₁-C₆ alkyl), and n′ is 3. In some such embodiments, the removal of P in step b) is performed with an organic or inorganic hydroxide, and the removal of P′ in step b) is performed with boron tribromide, hydroiodic acid, pyridine hydrochloride or pyridine hydrobromide. In some of the foregoing embodiments, the cyclization occurs during the removal of P′.

### DESCRIPTION OF THE INVENTION

In some embodiments, this invention provides compounds of the Formula I: 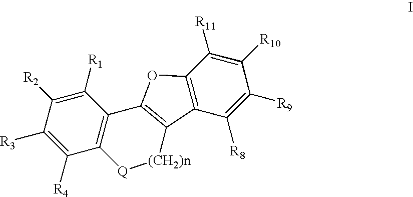
wherein:
Q has the structure II, III or IV: 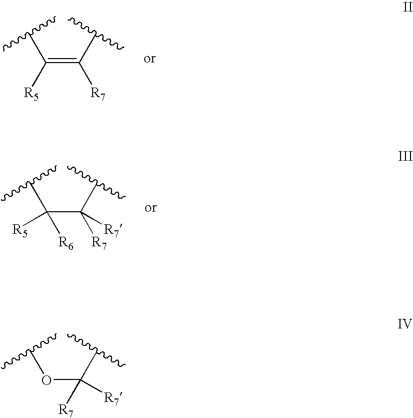
   R₁, R₄, R₅, R₆ R₇, R_{7′}, R₈ and R₁₁ are each independently selected from the group consisting of hydrogen, C₁-C₆ alkyl, —OR₂₀, halogen, —CF₃, —CF₂CF₃, —CH₂CF₃, —SR₂₀, NR₂OR₂₁, —CN, —CH₂CN, —CH₂CH₂CN, —CH═CHCN, —NO₂, —CH₂NO₂, —CH₂CH₂NO₂, —CH═CHNO₂ and —COR₂₀;
n=0 or 1;
   each R₂₀ and R₂₁ is independently selected from the group consisting of hydrogen, C₁-C₆ alkyl, —CF₃, benzyl, —CO₂(C₁-C₆alkyl) and —CO(C₁-C₆ alkyl); provided that:
   a) one of R₂ or R₃ must be —OR₂₀;
   b) one of R₉ or R₁₀ must be —OR₂₀;
   c) when R₂ is —OR₂₀, then R₁ and R₃ are independently selected from the group consisting of hydrogen, halogen, C₁-C₆ alkyl, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   d) when R₃ is —OR₂₀, then R₂ and R₄ are independently selected from the group consisting of hydrogen, C₁-C₆ alkyl, halogen, —CF₃, —CF₂CF₃, —CH₂CF₃′—SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   e) when R₉ is —OR₂₀, then R₈ and R₁₀ are independently selected from the group consisting of hydrogen, C₁-C₆ alkyl, halogen, —CF₃, —CF₂CF₃, —CH₂CF_{3′}—SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   f) when R₁₀ is —OR₂₀, then R₉ and R₁₁ are independently selected from the group consisting of hydrogen, C₁-C₆ alkyl, halogen, —CF₃, —CF₂CF₃, —CH₂CF₃′—SR₂₀, —CN, —CH₂CN, —CH₂CH₂CN, —CH═CHCN, —NO₂, —CH₂NO₂, —CH₂CH₂NO 2, —CH═CHNO₂ and —COR₂₀; and
   g) when Q has the structure IV, and R₇, R₇′, R₈, R₉, R₁₁ are each H, and n=0, then R₁₀ is not OR₂₀;
   or pharmaceutically acceptable salts thereof.

In some embodiments of the compounds of Formula I, Q has the structure II.

In some further embodiments of the compounds of Formula I, Q has the structure II, and R₃ and R₉ are each independently OR₂₀. In other embodiments of the compounds of Formula I, Q has the structure II, and R₃ and R₁₀ are each independently OR₂₀. In still other embodiments Q has the structure II and R₂ and R₉ are each independently OR₂₀. In still other embodiments, Q has the structure II and R₂ and R₁₀ are each independently OR₂₀.

In some embodiments of the compounds of Formula I, Q has the structure II where R₃ and R₉ are each independently OR₂₀; R₁, R₂, R₄, R₈ and R₁₀ are each independently hydrogen or halogen; and R₁₁ is CN, halogen, OCH₃, CH₂CN, NO₂ or C₁-C₆ alkyl.

In some embodiments of the compounds of Formula I, Q has the structure II wherein R₃ and R₉ are each independently OR₂₀; R₁, R₂, R₄, R₈ and R₁₀ are each independently hydrogen or halogen; R₁, is CN, halogen, OCH₃, CH₂CN, NO₂ or C₁-C₆ alkyl; and n is 0.

In some embodiments of the compounds of Formula I, Q has the structure II where R₃ and R₉ are each independently OR₂₀; R₁, R₂, R₄, R₈ and R₁₀ are each independently hydrogen or halogen; R₁₁ is CN, halogen, OCH₃, CH₂CN, NO₂ or C₁-C₆ alkyl; and n is 1.

In some embodiments of the compounds of Formula I, Q has the structure III. In some embodiments, Q has the structure III, and R₃ and R₉ are each independently OR₂₀. In some embodiments, Q has the structure III, and R₃ and R₁₀ are each independently OR₂₀. In yet other embodiments, Q has the structure III, and R₂ and R₉ are each independently OR₂₀. In other embodiments, Q has the structure III, and R₂ and R₁₀ are each independently OR₂₀.

In some embodiments of the compounds of Formula I, Q has the structure III; R₃ and R₉ are each independently OR₂₀; R₂, R₄, R₈ and R₁₀ are each independently hydrogen or halogen; and R₁₁ is CN, halogen, OCH₃, CH₂CN, NO₂ or C₁-C₆ alkyl. In some embodiments Q has the structure III; R₃ and R₉ are each independently OR₂₀; R₂, R₄, R₈ and R₁₀ are each independently hydrogen or halogen; and R₁₁ is CN, halogen, OCH₃, Me, CH₂CN, NO₂ or C₁-C₆ alkyl; and n is equal to 0. In some embodiments Q has the structure III; R₃ and R₉ are each independently OR₂₀; R₂, R₄, R₈ and R₁₀ are each independently hydrogen or halogen; R₁ is CN, halogen, OCH₃, CH₂CN, NO₂, or C₁-C₆ alkyl; and n is equal to 1.

In some embodiments of the compounds of Formula I, Q has the structure IV. In some embodiments, Q has the structure IV, and R₃ and R₉ are each independently OR₂₀. In some embodiments, Q has the structure IV, and R₃ and R₁₀ are each independently OR₂₀. In yet other embodiments, Q has the structure IV, and R₂ and R₉ are each independently OR₂₀. In further embodiments, Q has the structure IV, and R₂ and R₁₀ are each independently OR₂₀.

In some embodiments of the compounds of Formula I, Q has the structure IV; R₃ and R₉ are each independently OR₂₀; R₂, R₄, R₈ and R₁₀ are each independently hydrogen or halogen; and R₁₁ is CN, halogen, OCH₃, CH₂CN, NO₂ or C₁-C₆ alkyl. In some embodiments Q has the structure IV; R₃ and R₉ are each independently OR₂₀; R₂, R₄, R₈ and R₁₀ are each independently hydrogen or halogen; R₁₁ is CN, halogen, OCH₃, CH₂CN, NO₂ or C₁-C₆ alkyl; and n is equal to 0. In some embodiments Q has the structure IV; R₃ and R₉ are each independently OR₂₀; R₂, R₄, R₈ and R₁₀ are each independently hydrogen or halogen; R₁₁ is CN, halogen, OCH₃, CH₂CN, NO₂ or C₁-C₆ alkyl; and n is equal to 1.

In some embodiments, this invention provides compounds having the structure: 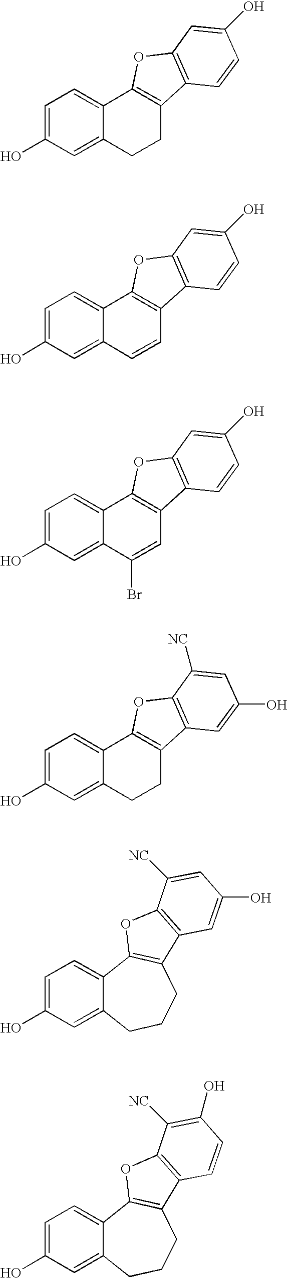 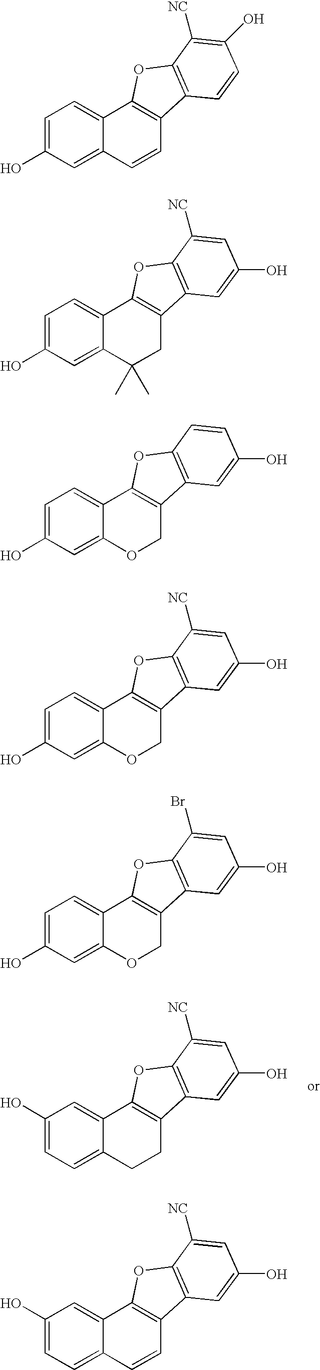
or pharmaceutically acceptable salts of each thereof.

The compounds of the invention are useful for treatment or prevention of symptoms of a variety of diseases and disorders in mammals that involve, relate to, or are affected by estrogenic agents. Nonlimiting examples of such diseases and disorders include treatment or inhibition of osteoporosis, inhibiting bone demineralization, inflammatory bowel disease, Crohn's disease, ulcerative proctitis, colitis, prostatic hypertrophy, uterine leiomyomas, breast cancer, polycystic ovary syndrome, endometrial polyps, benign breast disease, adenomyosis, ovarian cancer, melanoma, prostate cancer, colon cancer, glioma, astioblastomia, hypercholesteremia, hyperlipidemia, cardiovascular disease, atherosclerosis, peripheral vascular disease, restenosis, vasospasm, and vascular damage.

The compounds of the invention further find use in providing cognition enhancement or neuroprotection, treating or inhibiting senile dementias, Alzheimer's disease, cognitive decline, stroke, anxiety, or neurodegenrative disorders in a mammal, treating or inhibiting free radical induced disease states in a mammal, treating or inhibiting vaginal or vulvar atrophy, atrophic vaginitis, vaginal dryness, pruritus, dyspareunia, dysuria, frequent urination, urinary incontinence and urinary tract infections in a mammal, and treating or inhibiting vasomotor symptoms in a mammal.

The compounds of the invention also are useful for contraception, treating or inhibiting rheumatoid arthritis, osteoarthritis, or spondyloarthropathies in a mammal, treating or inhibiting joint damage secondary to arthroscopic or surgical procedures in a mammal, treating or inhibiting fertility in a mammal, treating or inhibiting ischemia, reperfusion injury, asthma, pleurisy, multiple sclerosis, systemic lupus erythematosis, uveitis, sepsis, hemorrhagic shock, or type II diabetes in a mammal, and lowering cholesterol, triglycerides, Lp(a), or LDL levels in a mammal.

This present invention further provides pharmaceutical compositions comprising one or more compounds of the invention, and one or more pharmaceutically acceptable carriers. In some embodiments, the pharmaceutical compositions include one or more of: 5,6-Dihydro-benzo[b]naphtho[2,1-d]furan-3,9-diol; benzo[b]naphtho[2,1-d]furan-3,9-diol; 5-bromo-benzo[b]naphtho[2,1-d]furan-3,9-diol; 3,8-dihydroxy-5,6-dihydro-benzo[b]naphtho[2,1-d]furan-10-carbonitrile; 3,9-dihydroxy-6,7-dihydro-5H-12-oxa-dibenzo[a,e]azulen-11-carbonitrile; 3,9-dihydroxy-5,6-dihydro-benzo[b]naphtho[2,1-d]furan-10-carbonitrile; 3,9-dihydroxy-benzo[b]naphtho[2,1-d]furan-10-carbonitrile; 3,8-dihydroxy-5,5-dimethyl-5,6-dihydro-benzo[b]naphtho[2,1-d]furan-10-carbonitrile; 6H-benzo[4,5]furo[3,2-c]chromen-3,8-diol; 3,8-dihydroxy-6H-Benzo[4,5]furo[3,2-c]chromene-10-carbonitrile; 10-bromo-6H-benzo[4,5]furo[3,2-c]chromene-3,8-diol; 2,9-dihydroxy-5,6-dihydro-benzo[b]naphtho[2,1-d]furan-10-benzonitrile; 2,9-dihydroxy-benzo[b]naphtho[2,1-d]furan-10-carbonitrile; and one or more pharmaceutically acceptable carriers.

The compounds of the invention can be prepared by coupling a compound of Formula V: 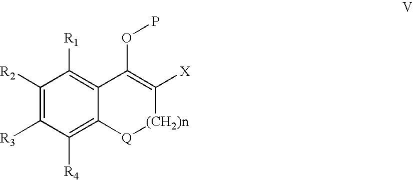
wherein X is Cl, Br, or I; and
P is a protecting group;
   with a compound of Formula VI 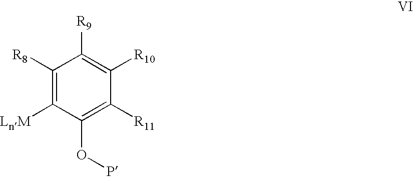
   wherein
M is a metal; and
L is a ligand; and
n′ is an integer from 0 to 5; and
P′ is H or a protecting group;
to form a compound of Formula VII; and 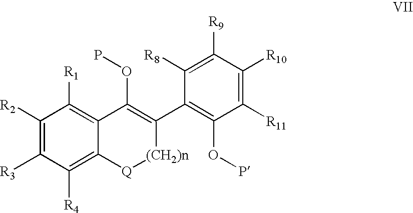
b) removing the groups P and P′ and cyclizing the resulting deprotected compound to form the compound of Formula I. 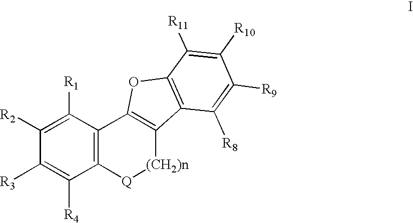
   wherein R₁, R₂, R₃, R₄, R₅, R₆, Q, n, R₇, R_{7′}, R₈, R₉, R₁₀, R₁₁ are as defined above.

In some embodiments of the process just described, P is Si(R′)₃, COC₁-C₆ alkyl, COOC₁-C₆ alkyl, CObenzyl, CO₂benzyl, or C₁-C₆ alkyl; each R′ is independently C₁-C₆ alkyl or phenyl; and P′ is H, Si(R′)₃, COC₁-C₆ alkyl, COOC₁-C₆ alkyl, CObenzyl, or C₁-C₆ alkyl; wherein each R′ is independently C₁-C₆ alkyl or phenyl. In other embodiments of the process just described, P is COC₁-C₆ alkyl, COOC₁-C₆ alkyl, CObenzyl, or CO₂benzyl; P′ is C₁-C₆ alkyl; and either: a) M is B, L is (OH) or (OC₁-C₆ alkyl), and n′ is 2; or b) M is Sn, L is (C₁-C₆ alkyl), and n′ is 3. In some such embodiments, the removal of P in step b) is performed with an organic or inorganic hydroxide, and the removal of P′ in step b) is performed with boron tribromide, hydroiodic acid, pyridine hydrochloride or pyridine hydrobromide. In some of the foregoing embodiments, the cyclization occurs during the removal of P′.

Compounds of this invention include pharmaceutically acceptable salts thereof wherein said pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts also may be formed from organic and inorganic bases, such as alkali metal salts (for example: sodium, lithium, or potassium), alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when a compound of this invention contains an acidic moiety.

As used herein, the term alkyl is intended to denote hydrocarbon groups, including straight chain, branched and cyclic hydrocarbons, including for example but not limited to methyl, ethyl, n-propyl, isopropyl, cyclopropyl, n-butyl, sec-butyl, tert-butyl, cyclobutyl, cyclopropylmethyl, n-pentyl, isopentyl, tert-pentyl, cyclopentyl, cyclopentylmethyl, n-hexyl, cyclohexyl, and the like. Throughout this specification, it should be understood that the term alkyl is intended to encompass both non-cyclic hydrocarbon groups and cyclic hydrocarbon groups. In some embodiments of the compounds of the invention, alkyl groups are non-cyclic. In further embodiments, alkyl groups are cyclic, and in further embodiments, alkyl groups are both cyclic and noncyclic.

Alkyl groups of the compounds and methods of the invention can include optional substitution with from one halogen up to perhalogenation. In some embodiments, perfluoro groups are preferred. Examples of alkyl groups optionally substituted with halogen include CF₃, CH₂CF₃, CCl₃, CH₂CH₂CF₂CH₃, CH(CF₃)₂, and (CH₂)₆—CF₂CCl₃.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, propyl, isopropyl, n-butyl, sec-butyl, isobutyl, etc. As used herein, the term halogen has its normal meaning of group VII elements, including F, Cl, Br and I.

Where compounds of the present methods can contain one or more asymmetric atoms, and thus give rise to optical isomers (enantiomers) and diastereomers, methods of the present invention include all such optical isomers (enantiomers) and diastereomers (geometric isomers); as well as the racemic and resolved, enantiomerically pure R and S stereoisomers; as well as other mixtures of the R and S stereoisomers or pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

As used in accordance with this invention, the term “providing,” with respect to providing a compound or substance covered by this invention, means either directly administering such a compound or substance, or administering a prodrug, derivative, or analog that will form the effective amount of the compound or substance within the body.

As will be appreciated from the standard pharmacological test procedure described below, the compounds of this invention are ER modulators useful in the treatment or inhibition of conditions, disorders, or disease states that are at least partially mediated by an estrogen deficiency or excess, or which may be treated or inhibited through the use of an estrogenic agent. The compounds of this invention are particularly useful in treating a peri-menopausal, menopausal, or postmenopausal patient in which the levels of endogenous estrogens produced are greatly diminished. Menopause is generally defined as the last natural menstrual period and is characterized by the cessation of ovarian function, leading to the substantial diminution of circulating estrogen in the bloodstream. As used herein, menopause also includes conditions of decreased estrogen production that may be caused surgically or chemically, or be caused by a disease state which leads to premature diminution or cessation of ovarian function.

Accordingly, the compounds of this invention are useful in treating or inhibiting osteoporosis and in the inhibition of bone demineralization, which may result from an imbalance in a individual's formation of new bone tissues and the resorption of older tissues, leading to a net loss of bone. Such bone depletion results in a range of individuals, particularly in post-menopausal women, women who have undergone bilateral oophorectomy, those receiving or who have received extended corticosteroid therapies, those experiencing gonadal dysgenesis, and those suffering from Cushing's syndrome. Special needs for bone replacement, including teeth and oral bone, also can be addressed using these compounds in individuals with bone fractures, defective bone structures, and those receiving bone-related surgeries and/or the implantation of prosthesis. In addition to those problems described above, these compounds can be used in treatment or inhibition for osteoarthritis, hypocalcemia, hypercalcemia, Paget's disease, osteomalacia, osteohalisteresis, multiple myeloma and other forms of cancer having deleterious effects on bone tissues.

The compounds of this invention also are useful in treating or inhibiting benign or malignant abnormal tissue growth, including prostatic hypertrophy, uterine leiomyomas, breast cancer, endometriosis, endometrial cancer, polycystic ovary syndrome, endometrial polyps, benign breast disease, adenomyosis, ovarian cancer, melanoma, prostrate cancer, cancers of the colon, and CNS cancers, such as glioma or astioblastomia.

The compounds of this invention are cardioprotective and they are useful in in lowering cholesterol, triglycerides, Lp(a), and LDL levels; inhibiting or treating hypercholesteremia, hyperlipidemia, cardiovascular disease, atherosclerosis, peripheral vascular disease, restenosis, and vasospasm, and in inhibiting vascular wall damage from cellular events leading toward immune mediated vascular damage. These cardiovascular protective properties are of great importance when treating postmenopausal patients with estrogens to inhibit osteoporosis and in the male when estrogen therapy is indicated.

The compounds of this invention also are antioxidants, and are therefore useful in treating or inhibiting free radical induced disease states. Specific situations in which antioxidant therapy is indicated to be warranted are with cancers, central nervous system disorders, Alzheimer's disease, bone disease, aging, inflammatory disorders, peripheral vascular disease, rheumatoid arthritis, autoimmune diseases, respiratory distress, emphysema, prevention of reperfusion injury, viral hepatitis, chronic active hepatitis, tuberculosis, psoriasis, systemic lupus erythematosus, adult respiratory distress syndrome, central nervous system trauma and stroke.

The compounds of this invention also are useful in providing cognition enhancement, and in treating or inhibiting senile dementias, Alzheimer's disease, cognitive decline, neurodegenerative disorders, providing neuroprotection or cognition enhancement.

The compounds of this invention also are useful in treating or inhibiting inflammatory bowel disease, ulcerative proctitis, Crohn's disease, colitis, and menopausal related conditions, such as vasomotor symptoms including hot flushes, vaginal or vulvar atrophy, atrophic vaginitis, vaginal dryness, pruritus, dyspareunia, dysuria, frequent urination, urinary incontinence, urinary tract infections, vasomotor symptoms, including hot flushes, myalgia, arthralgia, insomnia, irritability, and the like, and in male pattern baldness, skin atrophy, acne, type II diabetes, dysfunctional uterine bleeding, and infertility.

The compounds of this invention are useful in disease states where amenorrhea is advantageous, such as leukemia, endometrial ablations, chronic renal or hepatic disease or coagulation diseases or disorders.

The compounds of this invention can be used as a contraceptive agent, particularly when combined with a progestin.

The term active ingredient in the context of pharmaceutical compositions of the invention is intended to mean a component of a pharmaceutical composition that provides the primary pharmaceutical benefit, as opposed to an inactive ingredient, which would generally be recognized as providing no pharmaceutical benefit. The term pharmaceutical composition is intended to mean a composition comprising at least one active ingredient and at least one ingredient that is not an active ingredient (for example and not with limitation, a filler, dye, or a mechanism for slow release), whereby the composition is amenable to use for a specified, efficacious outcome in a mammal (for example, and not with limitation, a human).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds of this invention may be given at an oral dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration.

Such doses may be administered in any manner useful in directing the active compounds herein to the recipient's bloodstream, including orally, via implants, parenterally (including intravenous, intraperitoneal and subcutaneous injections), rectally, intranasally, vaginally, and transdermally.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including, surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation also may consist of administering the active ingredient in water or a fruit juice, containing appropriate solubilizers or emulsifiers as needed.

In some cases it may be desirable to administer the compounds directly to the airways in the form of an aerosol.

The compounds of this invention also may be administered parenterally or intraperitoneally. Solutions or suspensions of these active compounds as a free base or pharmacologically acceptable salt can be prepared in water suitably mixed with a surfactant such as hydroxy-propylcellulose. Dispersions also can be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient also may be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, also may be used.

It is appreciated that certain features of the invention, which are, for clarity, described in the context of separate embodiments, also can be provided in combination in a single embodiment. Conversely, various features of the invention which are, for brevity, described in the context of a single embodiment, also can be provided separately or in any suitable subcombination.

In some embodiments of the compounds, compositions and methods described herein, the compounds, compositions and methods exclude the compound 3,8-Dihydroxy-5,6-dihydro-benzo[b]naphtho[2,1-d]furan-10-carbonitrile.

The invention will be described in greater detail by way of specific examples. The following examples are offered for illustrative purposes and are not intended to limit the invention in any manner. Those of skill in the art will readily recognize a variety of noncritical parameters that can be changed or modified to yield essentially the same results.

### EXAMPLES

### Synthesis of Exemplary Compounds

Synthesis of the compounds described in the following Examples are described in Schemes 1 through 9 below. The chemical preparation methods described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), and mass spectrometry, or by chromatography such as high performance liquid chromatography (HPLC) or thin layer chromatography.

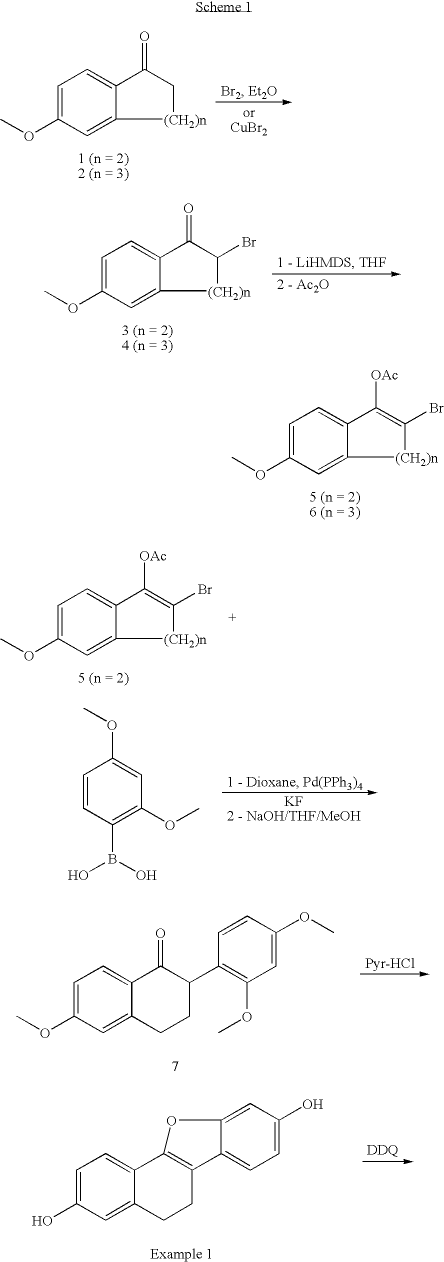

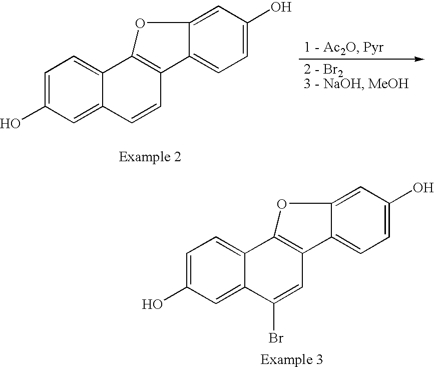

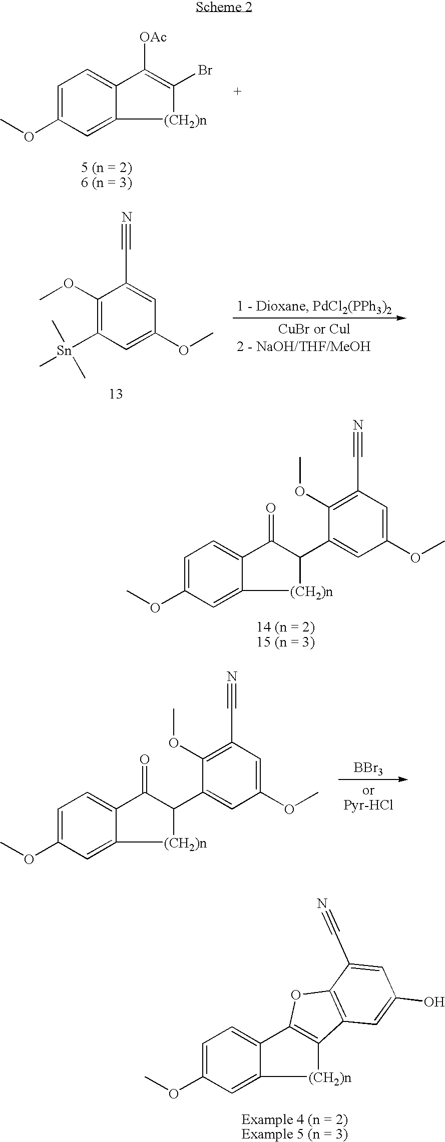

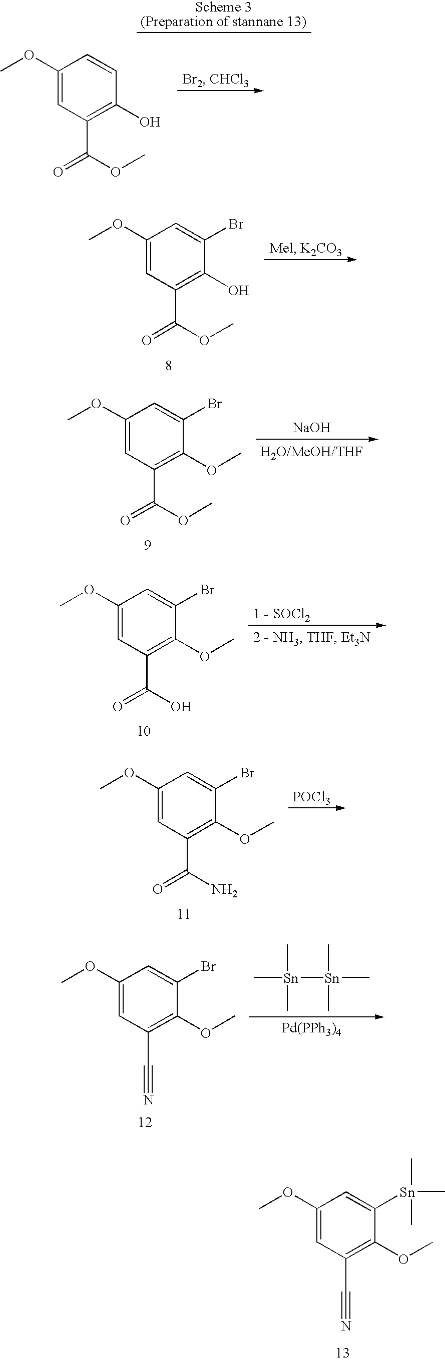

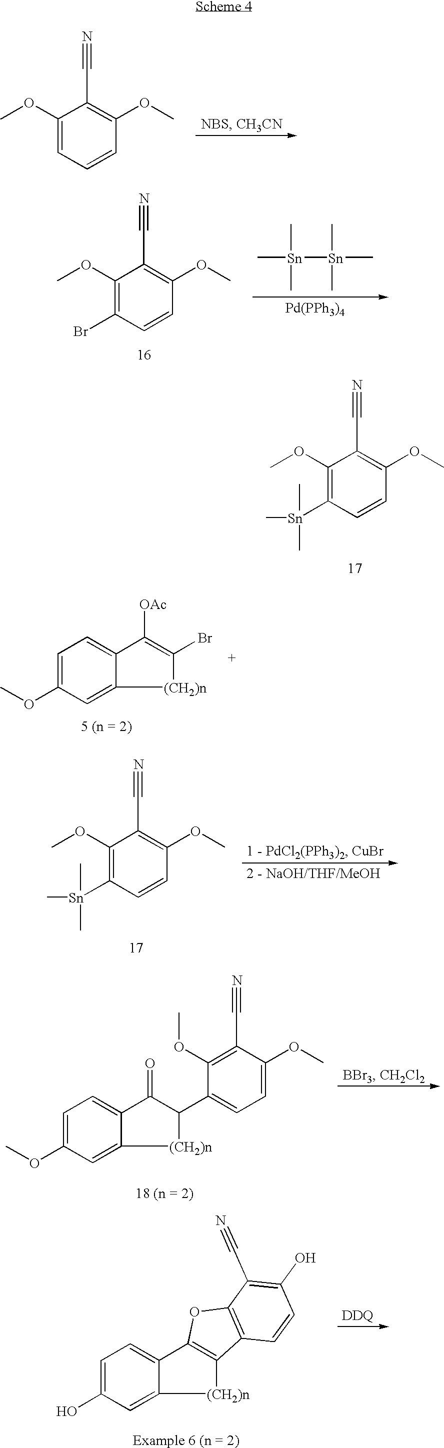

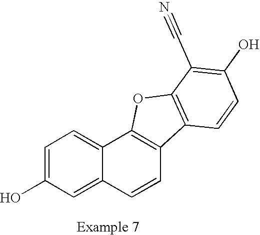

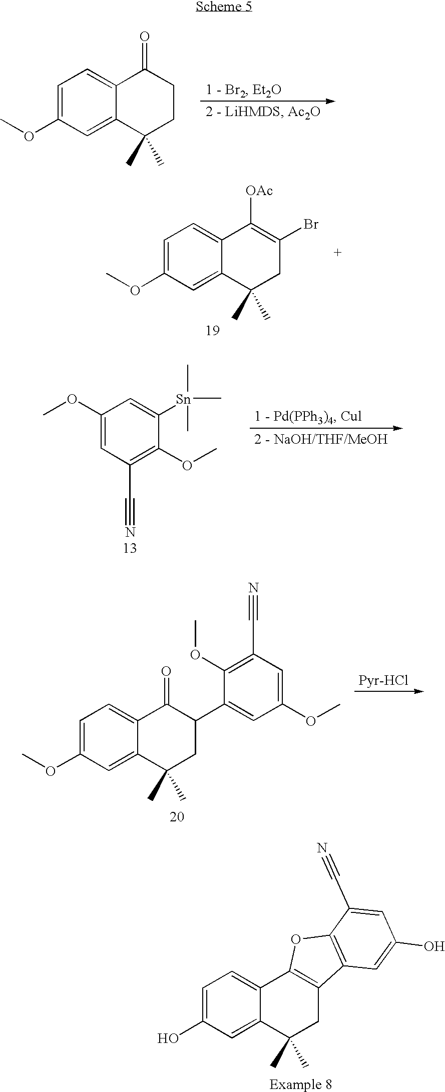

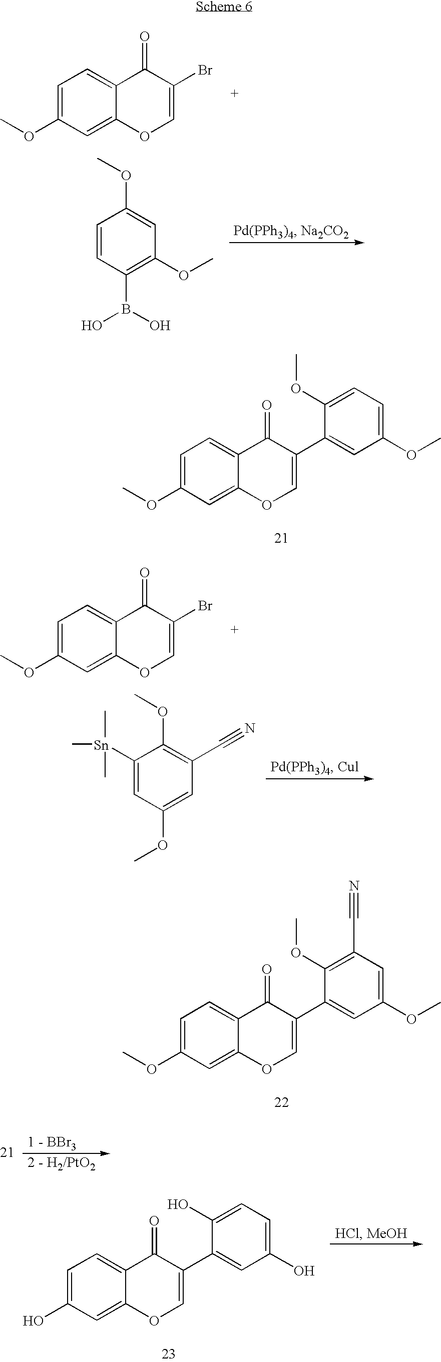

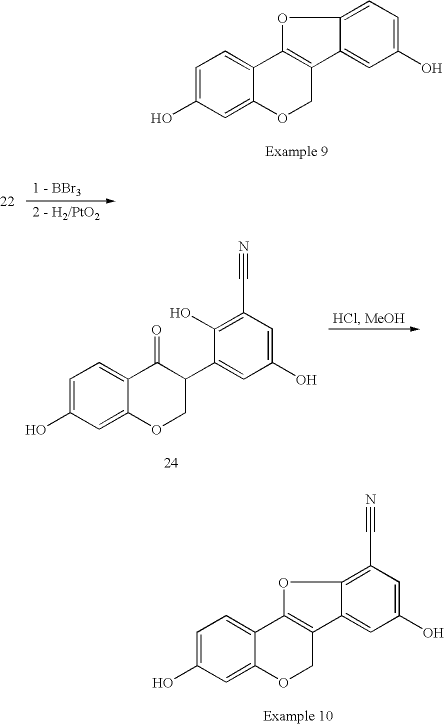

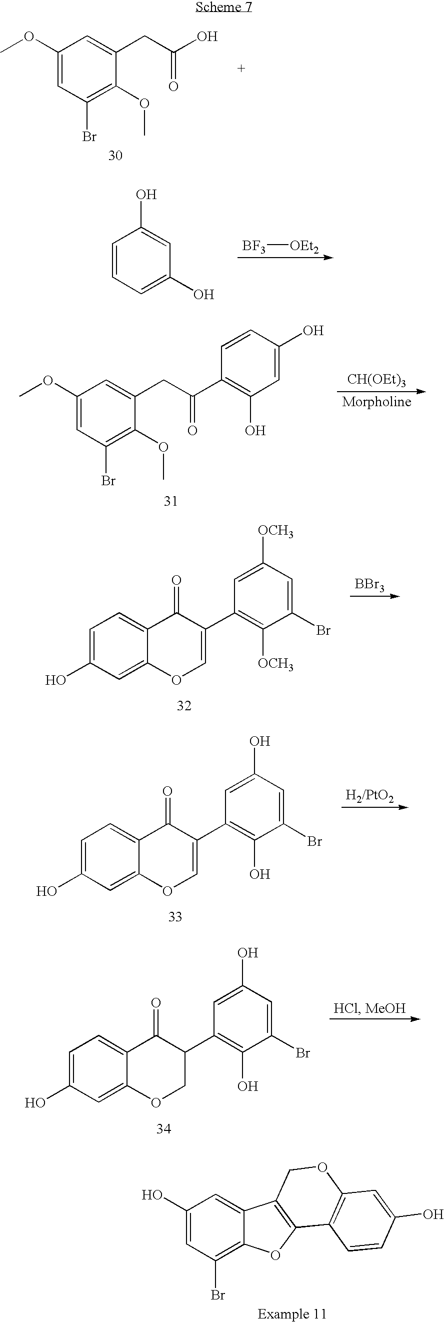

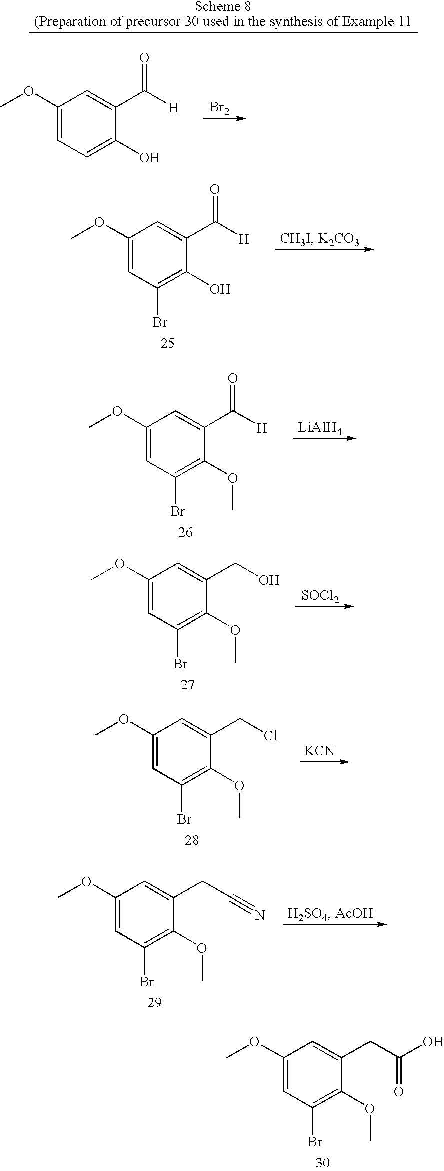

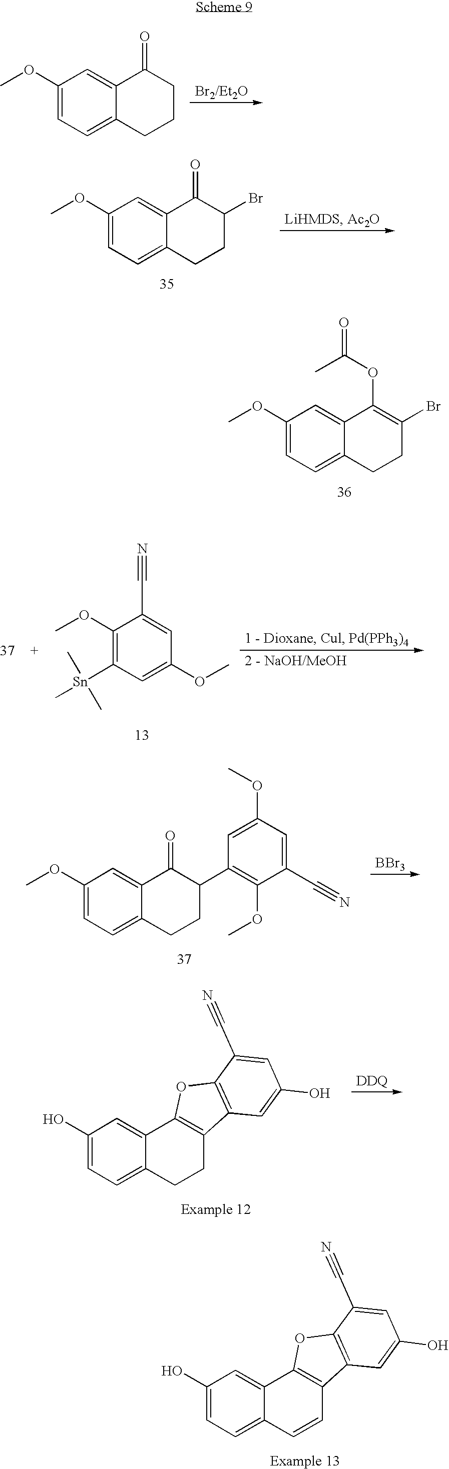

The preparation of representative examples of this invention is described below. Compound nomenclature was generated by inputting structures into ChemDraw® 5 or ChemDraw® Ultra and generating the name with the convert structure to name tool.

### Preparation of Examples 1, 2 and 3 (from Scheme 1)

### 2-BROMO-6-METHOXY-3,4-DIHYDRO-2H-NAPHTHALEN-1-ONE (3)

6-Methoxy-1-tetralone 1 (100 g, 0.567 mole) was dissolved in ethyl ether (2 liters) and treated with a dropwise addition of Br₂ (30 ml, 0.59 mole) over a 1 hour period. The solution was stirred for two additional hours and then worked up by washing with a 10% Na₂SO₃ solution, NaHCO₃ and brine. The solution was allowed to set overnight and 30 grams of crystals filtered off the following day. The remaining solution was concentrated to yield an additional 98 grams of product. The combined yield of the desired product was 128 g (88%). The material was used “as is” for subsequent reactions.

### ACETIC ACID 2-BROMO-6-METHOXY-3,4-DIHYDRO-NAPHTHALEN-1-YL ESTER (5)

A solution of 3 (80 g, 0.325 mole) in THF (200 mL) was cooled to −78° C. and treated with the slow addition of 0.65 liter of 0.53 molar LiHMDS in THF. The reaction was stirred for an additional 15 minutes at −78° C. and then treated with the rapid addition of acetic anhydride (100 g, 0.98 mole) in THF (200 mL). The reaction was stirred at 0° C. for 30 minutes and then worked up by diluting the reaction mixture with ethyl ether and washing with HCl (1 N), saturated NaHCO₃, water and brine. After drying over MgSO₄, the reaction was filtered and concentrated to give 83 grams of a dark oil that eventually solidified on standing: Mp (38-42° C.); ¹H NMR (CDCl₃) δ 7.00 (d, 1H, J=9.2 Hz), 6.71-6.88 (m, 2H), 3.79 (s, 3H), 3.00-2.84 (m, 4H), 2.34 (s, 3H).

### 2-(2,4-DIMETHOXY-PHENYL)-6-METHOXY-3,4-DIHYDRO-2H-NAPHTHALEN-1-ONE (7)

A solution of compound 5 (4.0 g, 0.014 mol) and 2,4-dimethoxy benzeneboronic acid (3.0 g, 0.016 mol), KF (4.0 g, 0.069 mol) and Pd(PPh₃)₄ (0.75 g, 0.0007 mol) was heated at reflux in dioxane (100 mL) overnight. The crude reaction mixture (after cooling to room temperature) was treated with a 50% NaOH (30 mL, aqueous) solution and stirred at room temperature until TLC indicated hydrolysis of the enol acetate was complete. The basic solution was neutralized with 2 N HCl and the dioxane removed under reduced pressure. The resultant mixture was extracted with ethyl acetate, washed with NaHCO₃, brine and dried over MgSO₄. Filtration, concentration and chromatography on silica gel (EtOAc/hexanes-gradient) yielded 7 as a white solid (2.9 g, 71%): Mp=116-118° C.; ¹H NMR (CDCl₃) δ 8.05 (d, 1H, J=8.7 Hz), 6.96 (d, 1H, J=8.2 Hz), 6.82 (dd, 1H, J=8.6 Hz, 2.1 Hz), 6.71 (s, 1H), 6.47 (d, 1H, J=2.1 Hz), 6.42 (d, 1H, J=8.2 Hz), 3.93 (dd, 1H, J=11.7 Hz, 4.6 Hz), 3.85 (s, 3H), 3.78 (s, 3H), 3.72 (s, 3H), 3.23-3.00 (m, 1H), 2.99-2.88 (m, 1H), 2.47-2.35 (m, 1H), 2.25-2.17 (m, 1H).

### 5,6-DIHYDRO-BENZO[B]NAPHTHO[2,1-D]FURAN-3,9-DIOL (EXAMPLE 1)

Compound 7 (1.5 g, 0.0048 mole) in Pyr-HCl was heated at 200° C. for 1 h. The reaction was allowed to cool to room temperature and worked up by partitioning between EtOAc and 2N HCl. The EtOAc layer was washed with NaHCO₃, brine and dried over MgSO₄. The solution was filtered, concentrated and chromatographed on silica gel (EtOAc/hexanes; 3:7 to 6:4). The product (Example 1) was contaminated with about 12% of the fully oxidized material (Example 2): Mp=219-220° C.; MS m/z 253 (M+H)⁺.

### BENZO[B]NAPHTHO[2,1-D]FURAN-3,9-DIOL

### Example 2

Example 1 (0.22 g, 0.00087 mole (based on 88% pure material)) was treated with DDQ (0.24 g, 0.001 mole) and heated to reflux in dioxane (20 mL) for 30 minutes. The reaction mixture was concentrated onto silica gel and chromatographed (EtOAc/hexanes; 3:7) to give Example 2 (0.1 g, 46%): Mp=250-260° C.; ¹H NMR (DMSO-d₆) δ 9.85 (s, 1H), 9.80 (s, 1H), 8.15 (d, 1H, J=8.9 Hz), 7.96 (d, 1H, J=8.6 Hz), 7.87 (d, 1H, J=8.3 Hz), 7.63 (d, 1H, J=8.6 Hz), 7.30 (d, 1H, J=1.9 Hz), 7.23 (dd, 1H, J=8.8 Hz, 2.1 Hz), 7.12 (d, 1H, J=1.9 Hz), 6.88 (dd, 1H, J=8.3 Hz, J=1.9 Hz).

### 5-BROMO-BENZO[B]NAPHTHO[2,1-D]FURAN-3,9-DIOL

### Example 3

A solution of Example 2 (0.25 g, 1.0 mmol) and pyridine (0.79 g, 10 mmol) in methylene chloride (10 ml) was treated with acetic anhydride (0.50 g, 5.0 mmol). After 2 h, the reaction was washed with 2N HCl, dried and concentrated to give the bis-acetylated intermediate as a white solid (0.28 g, 85%). A solution of the bis-acetate (0.28 g, 0.84 mmol) in methylene chloride (10 ml) was treated with Br₂ (0.15 g, 0.92 mmol). After 1 h, the reaction was washed with 10% sodium sulfite solution, dried and concentrated. The crude product was dissolved in THF (10 ml)/MeOH (2 ml) and 2N NaOH (1 ml) was added. After 1 h, the reaction was poured into 2N HCl and extracted with EtOAc. The organic layer was dried and concentrated to give a solid, which was triturated with CH₂Cl₂, then filtered to give Example 3 as a solid (0.13 g 47%); Mp=197-200° C.; ¹H NMR (DMSO-d₆) δ 10.27 (s, 1H), 9.93 (s, 1H), 8.47 (s, 1H), 8.23 (d, 1H, J=8.9 Hz), 7.93 (d, 1H, J=8.4 Hz), 7.56 (d, 1H, J=2.3 Hz), 7.30 (dd, 1H, J=8.8 Hz, 2.2 Hz), 7.14 (d, 1H, J=2.0 Hz), 6.89 (dd, 1H, J=8.5 Hz, 2.2 Hz).

### Preparation of Examples 4 and 5 (from Schemes 1 and 2)

### 6-BROMO-2-METHOXY-6,7,8,9-TETRAHYDRO-BENZOCYCLOHEPTEN-5-ONE (4)

2-Methoxy-6,7,8,9-tetrahydro-benzocyclohepten-5-one 2 (0.5 g, 2.62 mmol) was taken into 1:1 mixture of ethyl acetate and chloroform (10 mL), then CuBr₂ (1.17 g, 5.26 mmol) was added and the reaction was heated at 75° C. for 1 hour. The reaction was filtered and concentrated. The resulting material was taken into Et₂O and washed with water (2×), saturated NaHCO₃ (2×) and brine (1×). The ether layer was dried over MgSO₄, filtered and concentrated to yield 0.139 g (98.5%) of product 4 as a viscous liquid. ¹H NMR (CDCl₃) δ 7.69 (d, 1H, J=8.6 Hz), 6.81 (dd, 1H, J=8.6 Hz, 2.3 Hz), 6.71 (br s, 1H), 4.88 (dd, 1H, J=7.9 Hz, 4.2 Hz), 3.85 (s, 3H), 3.04 (m, 1H), 2.91 (m, 1H), 2.32 (m, 2H), 2.01 (m, 2H).

### ACETIC ACID 6-BROMO-2-METHOXY-8,9-DIHYDRO-7H-BENZOCYCLOHEPTEN-5-YL ESTER (6)

LiHMDS (9.98 mL of a 1 M solution in THF, 9.98 mmol) was taken into THF (10 mL) and cooled to −78° C. Then 6-bromo-2-methoxy-6,7,8,9-tetrahydro-benzocyclohepten-5-one 4 (2.44 g, 9.07 mmol) in THF (10 mL) was added dropwise and stirred for 20 minutes. Ac₂O in THF (2 mL) was added and stirred at 0° C. for 1 hour. The reaction was diluted with ether, then washed with 1 N HCl (2×), dilute NaHCO₃ and brine, and then dried over MgSO₄, filtered and concentrated to yield 3.0 g of product 6 as a yellow viscous liquid. ¹H NMR (CDCl₃) δ 7.15 (d, 1H, J=8.7 Hz), 6.69 (m, 2H), 3.74 (s, 3H), 2.74 (t, 2H, J=6.7 Hz), 2.49 (t, 2H, J=7.1 Hz), 2.17-2.11 (m, 5H).

### 2,5-DIMETHOXY-3-(6-METHOXY-1-OXO-1,2,3,4-TETRAHYDRO-NAPHTHALENE-2-YL)-BENZONITRILE (14)

To a solution of acetic acid 2-bromo-6-methoxy-3,4-dihydro-naphthalen-1-yl ester 5 (5.6 g, 19 mmol) and 2,5 dimethoxy-3-trimethylstannyl-benzonitrile 13 (7.0 g, 21 mmol) in dioxane under nitrogen was added copper bromide (0.15 g, 1.1 mmol) and dichlorobis(triphenylphosphine)palladium (0.74 g, 1.1 mmol), and this mixture was refluxed for 4 hours. The reaction was then cooled and 2N NaOH and methanol added. The reaction was warmed to about 40° C. and stirred several hours. The reaction was again cooled and then acidified with 2N HCl to pH2. The solvents were removed under reduced pressure and ethyl acetate added to the residue. This mixture was washed with saturated sodium bicarbonate and brine. The organic layer was dried over magnesium sulfate, concentrated and chromatographed on silica gel using ethyl acetate/hexane (1:9-3:7) to elute the product as a tan solid (1.2 g); ¹H (DMSO-d₆) δ 7.87 (d, 1H, J=9.4 Hz), 7.29 (d, 1H, J=3.1 Hz), 7.14 (d, 1H, J=3.1 Hz), 6.94-6.91 (m, 2H), 4.08 (dd, 1H, J=4.5 Hz, 13.3 Hz), 3.85 (s, 3H), 3.77 (s, 3H), 3.76 (s, 3H), 3.22-3.12 (m, 1H), 3.01-2.95 (m, 1H), 2.43 (dd, 1H, J=4.2 Hz, 13.0 Hz), 2.15-2.09 (m, 1H); MS ESI m/z 338 (M+H)⁺, 337 (M−H)⁻.

### 2,5-DIMETHOXY-3-(2-METHOXY-5-OXO-6,7,8,9-TETRAHYDRO-5H-BENZOCYCLOHEPTEN-6-YL)-BENZONITRILE (15)

Acetic acid 6-bromo-2-methoxy-8,9-dihydro-7H-benzocyclohepten-5-yl ester 6 (1.0 g, 3.21 mmol) was taken into dioxane (15 mL) along with CuI (0.061 g, 0.321 mmol), Pd(PPh₃)₄ (0.296 g, 0.257 mmol) and ⅓ the required amount of 2,5-dimethoxy-3-trimethylstannanyl-benzonitrile (−0.383 g of 1.15 g total, 3.53 mmol total). The remaining ⅔ of 2,5-dimethoxy-3-trimethylstannanyl-benzonitrile (0.767 g) was dissolved into dioxane (10 mL) and placed into an addition funnel. The reaction was heated at reflux for 30 minutes then 5 mL of the stannane/dioxane mixture was added and refluxed for another 30 minutes. Then the remaining 5 mL of the stannane/dioxane mixture was added and the reaction was refluxed overnight. TLC indicated that starting material was still present. Therefore, additional CuI (0.03 g) and Pd(PPh₃)₄ (0.074 g) was added and refluxing was continued for another 3 hours. To hydrolyze the acetate, an equal volume of 2 N NaOH was added along with THF and MeOH and the reaction was heated at 50° C. for 1 hour. 2 N HCl was added until pH 1 attained. The reaction mixture was concentrated and the resulting material was taken into EtOAc and washed with saturated NaHCO₃ (2×), brine (1×), dried over MgSO₄ and concentrated onto Florisil® for silica gel column chromatography (EtOAc/hexanes; 1:9 to 1:7). The product was isolated as 0.306 g of product as a yellow solid, and 0.130 g of this material was further purified by Prep HPLC (Luna® C18 (Phenomenex, Torrance, Calif.); 1:1 AcCN/H₂O to 95:5 AcCN/H₂O). ¹H NMR (DMSO-d₆) δ 7.60 (d, 1H, J=9.1 Hz), 7.27 (d, 1H, J=3.1 Hz), 7.19 (d, 1H, J=3.1 Hz), 6.93-6.90 (m, 2H), 4.26 (dd, 1H, J=11.6 Hz, 3.6 Hz), 3.84 (s, 3H), 3.79 (s, 3H), 3.71 (s, 3H), 3.16 (m, 1H), 2.97-2.91 (m, 1H), 2.16-2.08 (m, 2H), 1.90-1.86 (m, 1H), 1.70-1.66 (m, 1H); MS ESI m/z 352 [M+H]⁺.

### 3,8-DIHYDROXY-5,6-DIHYDRO-BENZO[B]NAPHTHO[2,1-D]FURAN-10-CARBONITRILE

### Example 4

To a solution of 2,5-dimethoxy-3-(6-methoxy-1-oxo-1,2,3,4-tetrahydro-naphthalen-2-yl)-benzonitrile 14 (0.5 g, 1.48 mmol) in dichloromethane was added 1.0M boron tribromide (10 mL, 10 mmol), which was stirred for 48 hours. The reaction was quenched with 2N HCl, the solvent was removed under reduced pressure and the residue partitioned between ethyl acetate and 2N HCl. The organic layer was dried over magnesium sulfate and concentrated. The residue was chromatographed on a Biotage® flash purification system (Uppsala, Sweden) using methanol/dichloromethane (2:98 to 3:97). The product fractions were combined and concentrated causing the precipitation of a yellow solid (0.16 g); Mp=355-358° C.; ¹H (DMSO-d₆) δ 9.88 (s, 1H), 9.81 (s, 1H), 7.43 (d, 1H, J=8.2 Hz), 7.20 (d, 1H, J=2.4 Hz), 7.03 (d, 1H, J=2.4 Hz), 6.77 (d, 1H, J=2.1 Hz), 6.73 (dd, 1H, J=2.4 Hz, 8.2 Hz), 3.01-2.95 (m, 2H), 2.86-2.80 (m, 2H); MS ESI m/z 278 (M+H)⁺, 276 (M−H)⁻.

### 3,9-DIHYDROXY-6,7-DIHYDRO-5H-12-OXA-DIBENZO[A,E]AZULEN-11-CARBONITRILE

### Example 5

2,5-Dimethoxy-3-(2-methoxy-5-oxo-6,7,8,9-tetrahydro-5H-benzocyclohepten-6-yl)-benzonitrile 15 (0.122 g, 0.347 mmol) was placed in a round-bottomed flask along with pyridine hydrochloride and heated at 200° C. for 1 hour. After cooling to room temperature, the solid was taken into an EtOAc/2 N HCl mixture. The layers were separated and the EtOAc layer was washed with 2 N HCl (2×) and dried over Mg SO₄. The product was purified by column chromatography on silica gel (EtOAc/hexanes: 1:3 to EtOAc/hexanes 1:2) to yield 0.048 g of product that still contained some impurity. The material was further purified using HPLC (5:95 ACN/H₂O to 95:5 ACN/H₂O) to yield 0.0127 g of pure product. ¹H NMR (DMSO-d₆) δ 9.84 (brs, 2H), 7.75 (d, 1H, J=8.6 Hz), 7.17 (d, 1H, J=2.3 Hz), 7.08 (d, 1H, J=2.3 Hz), 6.79 (dd, 1H, J=8.6 Hz, 2.6 Hz), 6.70 (d, 1H, J=2.3 Hz), 2.86 (m, 4H), 1.99 (m, 2H); MS ESI m/z 290 [M−H]⁻.

### PREPARATION OF STANNANE 13 (FROM SCHEME 3) 3-BROMO-2-HYDROXY-5-METHOXY-BENZOIC ACID METHYL ESTER (8)

To a solution of 5-methoxy salicylate methyl ester (20 mL, 0.13 mol) in chloroform was added bromine dropwise over 15 minutes. This mixture was stirred overnight at room temperature. The solvent was removed under reduced pressure to give 8 as a yellow solid (35 g). The product was used in subsequent steps without further purification; ¹H ((DMSO-d₆) δ 10.66 (s, 1H), 7.53 (d, 1H, J=3.0 Hz), 7.30 (d, 1H, J=3.0 Hz), 3.93 (s, 3H), 3.75 (s, 3H); MS ESI m/z 261 (M+H)⁺, 259 (M−H)⁻.

### 3-BROMO-2,5-DIMETHOXY-BENZOIC ACID METHYL ESTER (9)

To a solution of 3-bromo-2-hydroxy-5-methoxy-benzoic acid methyl ester 8 (˜35 g, 0.13 mol) in acetone was added methyl iodide (22.1 g, 0.156 mol) and potassium carbonate (36 g, 0.26 mol). This mixture was heated at reflux for 4 hours and then allowed to stir overnight at room temperature. The reaction was poured into water (500 mL), extracted into ether, dried over magnesium sulfate and concentrated to give 9 as a solid product (31.6 g); ¹H (DMSO-d₆) δ 7.45 (d, 1H, J=3.1 Hz), 7.22 (d, 1H, J=3.1 Hz), 3.86 (s, 3H), 3.78 (s, 3H), 3.75 (s, 3H).

### 3-BROMO-2,5-DIMETHOXY-BENZOIC ACID (10)

To a solution of 3-bromo-2,5-dimethoxy-benzoic acid methyl ester 9 (31.6 g, 115 mmol) in THF-methanol was added 50% NaOH (10 mL) and this mixture was heated at reflux for 4 hours and then the reaction was allowed to cool to room temperature and stirred overnight. The solvent was removed under reduced pressure and 2N HCl added until pH 1 was achieved and the mixture extracted with ethyl acetate. The organic layer was dried over magnesium sulfate and concentrated to render 10 as a white solid (27.8 g); ¹H (DMSO-d₆) δ13.50 (s, 1H), 7.40 (d, 1H, J=3.0 Hz), 7.20 (d, 1H, J=3.1 Hz), 3.77 (s, 3H), 3.75 (S, 3H); MS ESI m/z 259 (M−H)⁻.

### 3-BROMO-2,5-DIMETHOXY-BENZAMIDE (11)

3-Bromo-2,5-dimethoxy-benzoic acid 10 (27.7 g, 0.106 mol) was dissolved in thionyl chloride (155 mL, 2.12 mol) and to this solution was added a small amount of DMF (0.25 mL). This mixture was heated at reflux for 2 hours and then stirred at room temperature overnight. The thionyl chloride was removed under reduced pressure and replaced with THF. Then triethylamine (15 mL, 0.107 mol) was added and the reaction was cooled in an ice bath. Ammonia was bubbled into the mixture for about 8 minutes. The cooling bath was removed and the reaction was stirred at room temperature overnight. The solvent was removed under reduced pressure and the residue partitioned between ethyl acetate and 2N HCl. The organic layer was washed once with 2N HCl, then with saturated sodium bicarbonate and finally with brine. The organic layer was dried over magnesium sulfate and concentrated to give the crude product 11 (27 g); ¹H (DMSO-d₆) δ 7.78 (s, 1H), 7.64 (s, 1H), 7.30 (d, 1H, J=3.1 Hz), 7.08 (d, 1H, J=3.1 Hz), 3.77 (s, 3H), 3.73 (s, 3H); MS ESI m/z 260 (M+H)⁺.

### 3-BROMO-2,5-DIMETHOXY-BENZONITRILE (12)

To a solution of 3-bromo-2,5-dimethoxy-benzamide 11 (26.7 g, 0.103 mol) in THF was added phosphorous oxychloride (14 mL, 0.15 mol) and this mixture was heated at reflux overnight. The solvent was removed under reduced pressure and the residue partitioned between ethyl acetate and water. The organic layer was washed with saturated sodium bicarbonate and brine then dried over magnesium sulfate and concentrated. The residue was triturated with methanol to give an off-white product (19.6 g); ¹H (DMSO-d₆) δ 7.61 (d, 1H, J=3.0 Hz), 7.47 (d, 1H, J=3.0 Hz), 3.88 (s, 3H), 3.80 (s, 3H).

### 2,5-DIMETHOXY-3-TRIMETHYLSTANNANYL-BENZONITRILE (13)

To a solution of 3-bromo-2,5-dimethoxy-benzonitrile 12 (12.3 g, 51 mmol) in dioxane was added hexamethylditin (20 g, 61 mmol) and this mixture was purged with nitrogen. Then tetrakis(triphenylphosphine)palladium (3 g, 2.6 mmol) was added and the reaction heated at reflux for 6 hours and then allowed to cool to room temperature and stirred overnight. The solvent was removed under reduced pressure and the residue chromatographed on silica gel using ethyl acetate/hexane (3:97) to elute 13 as a white solid (11.9 g): Mp=74-76° C.; ¹H (DMSO-d₆) δ 7.31 (d, 1H, J=3.0 Hz), 7.17 (d, 1H, J=3.1 Hz), 3.86 (s, 3H), 3.77 (s, 3H), 0.31 (s, 9H).

### Preparation of Examples 6 and 7 (from Scheme 4)

### 3-BROMO-2,6-DIMETHOXY-BENZONITRILE (16)

To a solution of 2,6-dimethoxybenzonitrile (5 g, 31 mmol) in dichloromethane was added bromine in dichloromethane, dropwise over 1 hour. The reaction was stirred overnight at room temperature. The solvent was removed under reduced pressure to give the product 16 as a white solid (8.0 g) Mp=113-115° C. This material was used without further purification; ¹H (DMSO-d₆) δ 7.91 (d, 1H, J=9.2 Hz), 7.00 (d, 1H, J=9.1 Hz), 3.94 (s, 3H), 3.92 (s, 3H); MS ESI m/z 242 (M+H)⁺.

### 2,6-DIMETHOXY-3-TRIMETHYLSTANNANYL-BENZONITRILE (17)

To a solution of 3-bromo-2,6-dimethoxy-benzontrile 16 (5.8 g, 24 mmol) in dioxane under nitrogen was added hexamethylditin (10.0 g, 30.5 mmol) and tetrakis(triphenylphosphine)palladium (1.39 g, 1.2 mmol) and this mixture was refluxed for 24 hours. The reaction was concentrated and chromatographed on silica gel using ethyl acetate/hexane (1:9) to elute the product 17 (4.84 g); ¹H (DMSO-d₆) δ 7.58 (d, 1H, J=8.2 Hz), 6.97 (d, 1H, J=8.3 Hz), 3.92 (s, 3H), 3.89 (s, 3H), 0.28 (s, 9H); MS ESI m/z 326 (M+H)⁺.

### 2,6-DIMETHOXY-3-(6-METHOXY-1-OXO-1,2,3,4-TETRAHYDRO-NAPHTHALEN-2-YL)-BENZONITRILE (18)

To a solution of acetic acid 2-bromo-6-methoxy-3,4-dihydro-naphthalen-1-yl ester (4.0 g, 13.5 mmol) and 2,6-dimethoxy-3-trimethylstannyl-benzonitrile (4.84 g, 14.8 mmol) in dioxane under nitrogen was added copper bromide (106 mg, 0.74 mmol) and dichlorobis-(triphenylphosphine)palladium (520 mg, 0.74 mmol) and this mixture was refluxed for 2 hours. Then 2N NaOH (13.5 mL, 27 mmol) in methanol (10 mL) was added to the reaction and stirred for an hour. The reaction then was acidified to pH 6 via 2N HCl and the solvent was removed under reduced pressure and replaced with ethyl acetate. This mixture was washed with saturated sodium bicarbonate and brine. Then the organic layer was dried over magnesium sulfate, concentrated and chromatographed on silica gel using methanol/dichloromethane (2:98) to elute the product 18; ¹H (DMSO-d₆) δ 7.87 (d, 1H, J=9.4 Hz), 7.48 (d, 1H, J=8.8 Hz), 6.97-6.91 (m, 3H), 4.01 (dd, 1H, J=4.5 Hz, 13.1 Hz), 3.91 (s, 3H), 3.85 (s, 3H), 3.81 (s, 3H), 3.25-3.10 (m, 1H), 3.00-2.94 (m, 1H), 2.37 (dd, 1H, J=4.2 Hz, 12.9 Hz), 2.14-2.08 (m, 1H); MS ESI m/z 337 (M+H)⁺.

### 3,9-DIHYDROXY-5,6-DIHYDRO-BENZO[B]NAPHTHO[2,1-D]FURAN-10-CARBONITRILE

### Example 6

To a solution of 2,6-dimethoxy-3-(6-methoxy-1-oxo-1,2,3,4-tetrahydro-naphthalen-2-yl)benzonitrile (0.51 g, 1.5 mmol) in dichloromethane was added 1.0M BBr₃ (7.6 mL, 7.6 mmol) and this mixture was stirred at room temperature for 4 hours. The reaction was quenched with 2N HCl and the solvent removed under reduced pressure and replaced with ethyl acetate. This mixture was washed twice with 2N HCl and then once with brine. The organic layer was dried over magnesium sulfate, concentrated and chromatographed on silica gel using methanol/dichloromethane (1:99) to elute Example 6 as a tan solid (0.135 g): Mp>300° C.; ¹H (DMSO-d₆) δ 11.21 (s, 1H), 9.67 (s, 1H), 7.67 (d, 1H, J=8.6 Hz), 7.36 (d, 1H, J=8.2 Hz), 6.93 (d, 1H, J=8.6 Hz), 6.75 (d, 1H, J=2.2), 6.70 (dd, 1H, J=2.2 Hz, 8.2 Hz), 2.96 (t, 2H, J=7.6 Hz), 2.84 (t, 2H, J=8.0 Hz); MS ESI m/z 276 (M−H)⁻.

### 3,9-DIHYDROXY-BENZO[B]NAPHTHO[2,1-D]FURAN-10-CARBONITRILE

### Example 7

To a solution of 3,9-dihydroxy-5,6-dihydro-benzo[b]naphtho[2,1-d]furan-10-carbonitrile (Example 6 (51 mg, 0.19 mmol)) in dioxane was added DDQ (50 mg, 0.22 mmol) and this mixture was refluxed for an hour. The reaction was concentrated and chromatographed on silica gel using methanol/dichloromethane (5:95) to elute Example 7 as a white solid (18 mg): Mp>300° C.; ¹H (DMSO-d6) δ 11.55 (s, 1H), 10.00 (s, 1H), 8.18-8.24 (m, 2H), 8.03 (d, 1H, J=8.6 Hz), 7.72 (d, 1H, J=8.6 Hz), 7.34 (d, 1H, J=2.2 Hz), 7.28 (dd, 1H, J=2.3 Hz, 8.9 Hz), 7.08 (d, 1H, J=8.6 Hz); MS ESI m/z 274 (M−H)⁻.

### Preparation of Example 8 (from Scheme 5)

### Acetic acid 2-bromo-6-methoxy-4,4-dimethyl-3,4-dihydro-naphthalen-1-yl ester (19)

To a cooled solution of 6-methoxy-4,4-dimethyl-3,4-dihydro-2H-naphthalen-1-one (3.8 g 18.8 mmol) in ether (50 ml) was added bromine (0.96 ml, 18.6 mmol), dropwise. After 1 h, the reaction was washed with 10% aqueous sodium sulfite, dried and concentrated to give the bromide as a white solid (4.5 g), which was used crude. A portion of the resulting bromide (1.5 g, 5.3 mmol) was dissolved in THF (30 ml), cooled to −78° C. and treated with LHMDS (5.5 ml of 1M), dropwise. After 20 min, acetic anhydride (1.6 ml, 15.9 mmol) was added dropwise and the reaction was stirred at 0° C. for 1 h. Water was added and extracted with EtOAc. The EtOAc layer was dried, concentrated and the product was purified by column chromatography on silica gel (EtOAc/hexanes; 1:19) to give 19 as an oil (1.1 g).

### 2,5-DIMETHOXY-3-(6-METHOXY-4,4-DIMETHYL-1-OXO-1,2,3,4-TETRAHYDRO-NAPHTHALEN-2-YL)-BENZONITRILE (20)

Acetic acid 2-bromo-6-methoxy-4,4-dimethyl-3,4-dihydro-naphthalen-1-yl ester 19 (1 g, 3.1 mmol), 2,5-dimethoxy-3-trimethylstannyl-benzonitrile (1 g, 3.1 mmol), Pd(PPh₃)₄ (0.3 g) and CuI (50 mg) in dioxane (50 ml) was heated for 18 h. The reaction then was cooled and 1 N NaOH (5 ml) was added and the reaction was stirred for 1 h, then poured into water and extracted with EtOAc. The EtOAc layer was dried, concentrated and purified by column chromatography on silica gel (EtOAc/hexanes; 3:7) to give 20 as a yellow oil (0.25 g, 22%)

### 3,8-DIHYDROXY-5,5-DIMETHYL-5,6-DIHYDRO-BENZO[B]NAPHTHO[2,1-D]FURAN-10-CARBONITRILE

### Example 8

A mixture of 2,5-dimethoxy-3-(6-methoxy-4,4-dimethyl-1-oxo-1,2,3,4-tetrahydro-naphthalene-2-yl)-benzonitrile 20 (0.2 g, 0.55 mmol) and pyridine HCl (15 g) was heated to 200° C. After 1 h, the reaction was cooled and diluted with 2N HCl and extracted with EtOAc. The EtOAc layer was dried and concentrated to give a solid, which was purified by column chromatography (EtOAc/hexanes; 1:4) to give Example 8 as a white solid (35 mg, 21%): Mp=321-323° C.; ¹H NMR (DMSO-d₆) δ 9.85 (s, 2H), 7.46 (d, 1H, J=8.3 Hz), 7.19 (d, 1H, J=2.5 Hz), 7.03 (d, 1H, J=2.5 Hz), 6.88 (d, 1H, J=2.3 Hz), 6.73 (dd, 1H, J=8.2 Hz, 2.3 Hz), 2.76 (s, 2H), 1.28 (s, 6H).

### Preparation of Examples 9 and 10 (from Scheme 6)

### 3-(2,5-DIMETHOXY-PHENYL)-7-METHOXY-CHROMEN-4-ONE (21)

A solution of 3-bromo-7-methoxy-chromen-4-one (2.5 g, 10 mmol), 2,5-dimethoxyphenylboronoic acid (2.73 g, 15 mmol), 2M Na₂CO₃ (30 ml), and Pd(PPh₃)₄ (0.30 g, 0.3 mmol) in toluene (40 ml) and EtOH (5 ml) was heated to reflux. After 3 h the reaction was cooled, and the organic layer was separated, dried, and concentrated to give an oily solid, which was triturated with MeOH and filtered to give 21 as a white solid (1.5 g, 51%).

### 2,5-DIMETHOXY-3-(7-METHOXY-4-OXO-4H—CHROMEN-3-YL)-BENZONITRILE (22)

A solution of 3-bromo-7-methoxy-chromen-4-one (1.8 g, 7.1 mmol), 2,5-dimethoxy-3-trimethylstannyl-benzonitrile (2.3 g, 7.1 mmol), Pd(PPh₃)₄ (0.5 g), and CuI (0.1 g) in 50 mL dioxane was heated to reflux. After 6 h the reaction was cooled and concentrated and the product was purified by column chromatography on silica gel (EtOAc/hex; 1:4) to give 22 as a solid (0.9 g, 38%).

### 3-(2,5-DIHYDROXY-PHENYL)-7-HYDROXY-CHROMAN-4-ONE (23)

To a solution of 3-(2,5-dimethoxyphenyl)-7-methoxy-chromen-4-one 21 (1.5 g, 4.8 mmol) in CH₂Cl₂ (30 ml) was added BBr₃ (25 ml of 1M), dropwise. After stirring for 20 h, the reaction was cooled and carefully quenched with MeOH. The solution was diluted with EtOAc and washed with 2N HCl. The EtOAc layer was dried and concentrated to give a solid (1.1 g), which was taken up into acetone and hydrogenated over PtO₂ (0.18 g) at 10 psi. After 3 h, the reaction was filtered through Celite® and concentrated to give a foam. The foam was purified by column chromatography on silica gel (EtOAc/hexane; 1:4) to give 23 also as a foam (0.4 g, 31%).

### 2,5-DIHYDROXY-3-(7-HYDROXY-4-OXO-CHROMAN-3-YL)-BENZONITRILE (24)

A mixture of 2,5-dimethoxy-3-(7-methoxy-4-oxo-4H-chromen-3-yl)-benzonitrile 22 (0.90 g, 2.7 mmol) and pyridine HCl (15 g) was heated to 200° C. After 1 h the reaction was cooled and diluted with 2N HCl. The acidic layer then was extracted with EtOAc, dried, and concentrated, and the product was purified by column chromatography on silica gel (EtOAc/hexanes; 3:2) to give a solid (300 mg), which was taken up into acetone and hydrogenated over PtO₂ at 10 psi. After 1.5 h, the reaction was filtered, concentrated, and purified by column chromatography on silica gel to give 24 as a foam (0.15 g, 19%).

### 6H-BENZO[4,5]FURO[3,2-C]CHROMEN-3,8-DIOL

### Example 9

A solution of 3-(2,5-dihydroxy-phenyl)-7-hydroxy-chroman-4-one 23 (0.35 g, 1.25 mmol) in saturated HCl/MeOH (20 ml) was heated to reflux. After 1 h the reaction was cooled, concentrated and the product was purified by column chromatography on silica gel (EtOAc/hexanes; 3:7) to give Example 9 as a solid (80 mg, 25%): Mp=238-240° C.; ¹H NMR (DMSO-d₆) δ9.83 (s, 1H), 9.24 (s, 1H), 7.37 (d, 1H, J=8.8 Hz), 7.29 (d, 1H, J=8.8 Hz), 6.79 (d, 1H, J=1.8 Hz), 6.70 (d, 1H, J=7.7 Hz), 6.45 (d, 1H, J=7.2 Hz), 6.37 (s, 1H), 5.50 (s, 2H).

### 3,8-DIHYDROXY-6H-BENZO[4,5]FURO[3,2-C]CHROMENE-10-CARBONITRILE

### Example 10

A solution of 2,5-dihydroxy-3-(7-hydroxy-4-oxo-chroman-3-yl)-benzonitrile 24 (0.14 g, 0.47 mmol) in saturated HCl/MeOH (10 ml) was heated to reflux. After 1 h the reaction was cooled and a solid crystallized and it was collected by filtration to give Example 10 as a solid (60 mg, 43%): Mp>300° C.; ¹H NMR (DMSO-d₆) δ 9.99 (s, 2H), 7.35 (d, 1H, J=8.3 Hz), 7.18 (d, 1H, J=2.4 Hz), 7.07 (d, 1H, J=2.3 Hz), 6.48 (dd, 1H, J=8.3 Hz, 2.1 Hz), 6.40 (d, 1H, J=2.3 Hz), 5.53 (s, 2H).

### Preparation of Example 11 (from Scheme 7)

### 2-(3-Bromo-2,5-dimethoxy-phenyl)-1-(2,4-dihydroxy-phenyl)-ethanone (31)

A solution of (3-bromo-2,5-dimethoxy-phenyl)-acetic acid 30 (10 g, 36 mmol) and resorcinol (6.0 g, 54 mmol) in BF₃-etherate (75 ml) was heated to 85° C. After 4 h the reaction was cooled and poured on ice. The aqueous layer then was extracted with EtOAc. The EtOAc layer was dried and concentrated to give 31 as an orange oil (15 g), which was used crude for the next step.

### 3-(3-BROMO-2,5-DIMETHOXY-PHENYL)-7-HYDROXY-CHROMEN-4-ONE (32)

A mixture of 2-(3-bromo-2,5-dimethoxy-phenyl)-1-(2,4-dihydroxy)-ethanone 31 (15 g crude), triethylorthoformate (40 ml), and morpholine (40 ml) was heated to reflux. After 2 h, the reaction was cooled and poured into 2N HCl and extracted with EtOAc. The EtOAc layer was dried and concentrated and the resulting product was purified by column chromatography on silica gel (EtOAc/hexanes; 3:7) to give 32 as a solid (4 g, 30% over two steps).

### 3-(3-BROMO-2,5-DIHYDROXY-PHENYL)-7-HYDROXY-CHROMEN-4-ONE (33)

To a solution of 3-(3-bromo-2,5-dimethoxy-phenyl)-7-hydroxy-chromen-4-one 32 (4 g, 10.6 mmol) in CH₂Cl₂ (100 ml) was added BBr₃ (30 ml, 1M), dropwise. After 2 h, the reaction was cooled to 0° C. and carefully quenched with MeOH. The reaction was diluted with EtOAc and washed with 2N HCl. The EtOAc layer was dried and concentrated to give a dark solid, which was triturated with MeOH and filtered to give 33 as a solid (2.7 g, 73%); Mp=253-255° C.; ¹H NMR (DMSO-d₆) δ 10.86 (s, 1H), 9.26 (s, 1H), 8.59 (s, 1H), 8.28 (s, 1H), 7.96 (d, 1H, J=8.7 Hz), 6.98-6.90 (m, 3H), 6.62 (d, 1H, J=2.9 Hz).

### 3-(3-BROMO-2,5-DIHYDROXY-PHENYL)-7-HYDROXY-CHROMAN-4-ONE (34)

A solution of 33 (1.5 g, 4.3 mmol) in acetone (40 ml) was hydrogenated over PtO₂ (0.25 g) at 10 psi. After 3 h, the reaction was filtered through Celite® and concentrated to give a foam, which was purified by column chromatography on silica gel (EtOAc/hexanes; 1:3) to give 34 as a foam (1 g, 66%).

### 10-BROMO-6H-BENZO[4,5]FURO[3,2-C]CHROMENE-3,8-DIOL

### Example 11

3-(3-Bromo-2,5-dihydroxy-phenyl)-7-hydroxy-chroman-4-one 34 (0.95 g, 2.7 mmol) in saturated HCl/MeOH was heated to reflux. After 30 min, the reaction was concentrated, taken up into EtOAc and washed with saturated NaHCO₃. The EtOAc was dried and concentrated to give an oily solid, which was triturated with CH₂Cl₂ and filtered to give Example 11 as a solid (0.6 g, 66%); Mp=222-225° C.; ¹H NMR (DMSO-d₆) δ 9.92 (s, 1H), 9.65 (s, 1H), 7.30 (d, 1H, 8.3 Hz), 6.91 (d, 1H, J=2.2 Hz), 6.83 (d, 1H, J=2.2 Hz), 6.45 (dd, 1H, J=8.3 Hz, 1.7 Hz), 6.38 (d, 1H, J=1.9 Hz), 5.50 (s, 2H).

### Preparation of Precursor 30 (from Scheme 8)

### 3-BROMO-2-HYDROXY-5-METHOXY-BENZALDEHYDE (25)

To a cooled 0° C. solution of methyl 4-methoxysalicylate (30 g, 200 mmol) in chloroform (500 ml) was added bromine (32 g, 200 mmol) and the reaction was stirred at room temperature for 5 hr. The reaction then was washed with 10% sodium sulfite, dried, and concentrated to give a solid. The solid was triturated with hexane and filtered to give 25 as a yellow solid (14 g, 35%): Mp=107-110° C.

### 3-BROMO-2,5-DIMETHOXY-BENZALDEHYDE (26)

A solution of 25 (10 g, 43 mmol), methyl iodide (7.3 g, 52 mmol), and K₂CO₃ (12 g, 86 mmol) in acetone (200 ml) was heated to reflux. After 4 hr, the reaction was cooled, poured into water and extracted with ether. The ether layer was dried and concentrated, and the product was purified by silica gel column chromatography (10% EtOAc/hex) to give 26 as a solid (7.0 g, 67%): Mp=62-64° C.; ¹H NMR (CDCl₃) δ 10.32 (s, 1H), 7.38 (d, 1H, J=2.8 Hz), 7.28 (d, 1H, J=3.2 Hz), 3.93 (s, 3H), 3.82 (s, 3H); MS ESI m/z 245/247 (M+H)⁺

### (3-BROMO-2,5-DIMETHOXY-PHENYL)-METHANOL (27)

To a cooled (0° C.) solution of 26 (8.0 g, 33 mmol) in THF (100 ml) was added LiAlH₄ (15 ml of 1.0M in THF), dropwise. After 15 min, the reaction was quenched with 2N HCl and the aqueous layer was extracted with EtOAc. The EtOAc layer was dried and concentrated to give 27 as a solid (7.5 g, 93%): Mp=65-67° C.; ¹H NMR (DMSO-d₆) δ 7.05 (d, 1H, J=3.0 Hz), 6.98 (d, 1H, J=2.5 Hz), 5.28 (t, 1H, J=4.9 Hz), 4.47 (d, 2H, J=5.7 Hz), 3.73 (s, 3H), 3.67 (s, 3H); MS ESI m/z 245 (M−H)⁻.

### 1-BROMO-3-CHLOROMETHYL-2,5-DIMETHOXY-BENZENE (28)

To a solution of 27 (7.5 g, 30 mmol) and ZnCl₂ (1 g) in THF (100 ml) was added SOCl₂ (5.31 g, 45 mmol), dropwise. After 1 hr at room temperature, the reaction was poured into water and extracted with ether. The ether was dried, concentrated and the product was purified by column chromatography on silica gel (10% EtOAc/hex) to give 28 as an oil (5.5 g, 75%): ¹H NMR (DMSO-d₆) δ 7.21 (d, 1H, J=3.0 Hz), 7.08 (d, 1H, J=3.0 Hz), 4.73 (s, 2H), 3.78 (s, 3H), 3.75 (s, 3H).

### (3-BROMO-2,5-DIMETHOXY-PHENYL)-ACETONITRILE (29)

A solution of 1-bromo-3-chloromethyl-2,5-dimethoxy-benzene 28 (7.0 g, 26.4 mmol) and KCN (1.7 g, 26.4 mmol) in DMSO (50 ml) was heated to 75° C. After 2 hr, the reaction was cooled and poured into water. The aqueous layer was extracted with EtOAc and the organic layer was dried and concentrated. The product was purified by column chromatography on silica gel (20% EtOAc/Hex) to give 29 as an oil (5.2 g, 77%): ¹H NMR (DMSO-d₆) δ 7.20 (d, 1H, J=3.0 Hz), 6.99 (d, 1H, J=3.0 Hz), 4.00 (s, 2H), 3.75 (s, 6H).

### (3-BROMO-2,5-DIMETHOXY-PHENYL)-ACETIC ACID (30)

A solution of (3-bromo-2,5-dimethoxy-phenyl)-acetonitrile 29 (5.2 g, 20.4 mmol) in water (10 ml), conc. H₂SO₄ (10 ml), and AcOH (30 ml) was heated to 100° C. After 3 hr, the reaction was cooled and poured into water. The aqueous layer was extracted with EtOAc, which was then dried over MgSO₄, filtered and concentrated. The product was purified by column chromatography on silica gel (50% EtOAc/Hex) to give 30 as a solid (2.8 g, 55%): Mp=62-65° C.; ¹H NMR (DMSO-d₆) δ 12.45 (br s, 1H), 7.09 (d, 1H, J=2.9 Hz), 6.87 (d, 1H, J=3.0 Hz), 3.72 (s, 3H), 3.66 (s, 3H), 3.59 (s, 2H); MS ESI m/z 273/275 (M−H).

### Preparation of Examples 12 and 13 (from Scheme 9)

### 2-BROMO-7-METHOXY-3,4-DIHYDRO-2H-NAPHTHALEN-1-ONE (35)

To a solution of 7-methoxy-1-tetralone (50 g, 0.28 mol) in ether was added bromine (15 mL, 0.29 mol), dropwise over 2 h. This solution was stirred an additional 2 h, then washed with 10% sodium sulfite, saturated sodium bicarbonate and brine. The organic layer was dried over MgSO₄ and concentrated until a white crystalline product 35 precipitated, which was collected by suction filtration (60.5 g); ¹H (DMSO-d₆) δ 7.39 (d, 1H, J=2.8 Hz), 7.34 (d, 1H, J=8.5 Hz), 7.22 (dd, 1H, J=2.8 Hz, 8.5 Hz), 5.03 (dd, 1H, J=3.6 Hz, 5.8 Hz), 3.80 (s, 3H), 3.10-2.85 (m, 2H), 2.60-2.50 (m, 1H), 2.40-2.28 (m, 1H).

### ACETIC ACID 2-BROMO-7-METHOXY-3,4-DIHYDRO-NAPHTHALEN-1-YL ESTER (36)

A solution of lithium bis(trimethylsilyl)amide (50 mL, 50 mmol) in THF was cooled to −78° C., under nitrogen, and to this was added 2-bromo-7-methoxy-3,4-dihydro-2H-naphthalen-1-one 35 (11.6 g, 45 mmol) dissolved in THF, dropwise over 30 minutes. This mixture was stirred 30 minutes and then acetic anhydride (12.8 mL, 135 mmol) was added dropwise over 10-15 minutes. The dry ice-acetone cooling was removed and replaced with an ice bath and the reaction stirred at 0° C. for an hour. The reaction was diluted with ether, washed with 1N HCl (3×25 mL) and then, once each, with dilute sodium bicarbonate, water and brine. The organic layer was dried over MgSO₄ and concentrated to yield 36 as a viscous liquid (13.2 g); ¹H (DMSO-d₆) δ7.14 (d, 1H, J=8.3 Hz), 6.84 (dd, 1H, J=2.6 Hz, 8.3 Hz), 6.65 (d, 1H, J=2.6 Hz), 3.73 (s, 3H), 2.87-2.84 (m, 4H), 2.36 (s, 3H).

### 2,5-DIMETHOXY-3-(7-METHOXY-1-OXO-1,2,3,4-TETRAHYDRONAPHTHALEN-2-YL)-BENZONITRILE (37)

To a solution of acetic acid 2-bromo-7-methoxy-3,4-dihydro-naphthalen-1-yl ester 36 (2.5 g, 8.4 mmol) and 2,5-dimethoxy-3-trimethylstannyl-benzonitrile (3.0 g, 9.3 mmol) in dioxane was added copper iodide (0.16 g, 0.84 mmol) and this mixture was refluxed overnight. The reaction was cooled and 2N NaOH (8.4 mL, 16.8 mmol) in methanol was added to the reaction, which was warmed to 40° C. for about an hour until hydrolysis of the acetate was complete (followed by TLC). The reaction mixture was acidified via 2N HCl, the solvents removed under reduced pressure and ethyl acetate added. This mixture was washed with saturated sodium bicarbonate and brine, the organic layer dried over magnesium sulfate, concentrated and chromatographed on silica gel using ethyl acetate/hexane (5:95 to 1:9) to elute 37 (0.6 g); ¹H (DMSO-d₆) δ 7.38-7.29 (m, 3H), 7.22-7.16 (m, 2H), 4.12 (dd, 1H, J=4.2 Hz, 13.3 Hz), 3.79 (s, 3H), 3.78 (s, 3H), 3.77 (s, 3H), 3.17-2.98 (m, 2H), 2.50-2.40 (m, 1H), 2.20-2.10 (m, 1H); MS ESI m/z 338 (M+H)⁺.

### 2,9-DIHYDROXY-5,6-DIHYDRO-BENZO[B]NAPHTHO[2,1-D]FURAN-10-BENZONITRILE (EXAMPLE 12)

To a solution of 2,5-dimethoxy-3-(7-methoxy-1-oxo-1,2,3,4-tetrahydronaphthalen-2-yl)-benzonitrile 37 (0.27 g, 0.8 mmol) in dichloromethane, under nitrogen, was added 1.0M BBr₃ (4.0 mL, 4 mmol) and this mixture was stirred at room temperature overnight. The reaction was quenched with 2N HCl, the solvent removed under reduced pressure and the residue partitioned between ethyl acetate and 2N HCl. The organic layer was dried over MgSO₄, concentrated and chromatographed on silica gel using ethyl acetate/hexane (1:3) to elute the product as an off-white solid (115 mg): Mp=277-279° C.; ¹H (DMSO-d₆) δ 9.94 (s, 1H), 9.50 (s, 1H), 7.26 (d, 1H, J=2.3 Hz), 7.15 (d, 1H, J=8.2), 7.12 (d, 1H, J=2.5 Hz), 7.04 (d, 1H, J=2.5 Hz), 6.68 (dd, 1H, J=2.5 Hz, 8.1 Hz), 2.90 (m, 4H); MS ESI m/z 278 (M+H)⁺.

### 2,9-DIHYDROXY-BENZO[B]NAPHTHO[2,1-D]FURAN-10-CARBONITRILE

### Example 13

To a solution of 2,9-dihydroxy-5,6-dihydro-benzo[b]naphtho[2,1-d]furan-10-benzonitrile (Example 12 (95 mg, 0.34 mmol)) in dioxane was added DDQ (93 mg, 0.41 mmol) and this mixture was refluxed for 4 hours. The solvent was removed under reduced pressure and the residue chromatographed on silica gel using methanol/dichloromethane (1:4) to elute the product as a brown solid (0.073 g): Mp=291-295° C.; ¹H (DMSO-d₆) δ 10.18 (s, 1H), 10.14 (s, 1H), 7.99 (d, 1H, J=8.9 Hz), 7.95 (d, 1H, J=8.5 Hz), 7.85 (d, 1H, J=2.4 Hz), 7.82 (d, 1H, J=8.5 Hz), 7.59 (d, 1H, J=2.2 Hz), 7.33 (d, 1H, J=2.4 Hz), 7.21 (dd, 1H, J=2.4 Hz, 8.9 Hz); MS ESI m/z 274 (M−H)⁻.

Evaluation of Compounds of the Invention

Representative examples of the invention were evaluated for their ability to compete with 17β-estradiol for both ERα and ERβ. This test procedure provides the methodology for one to determine whether a particular compound binds to the ER (and is therefore, “estrogenic”) and whether there is selectivity for ERα or ERβ. The values are shown in the Table, infra, and are reported as IC₅₀s. 17β-Estradiol is included as a standard reference for comparison. The procedure used is described briefly below. A crude lysate of E. coli expressing the ER ligand binding domains (D, E, & F) of human ERα or ERβ was prepared. Both ERs and compounds were diluted in 1× Dulbecco's Phosphate Buffered Saline (DPBS) supplemented with 1 mM EDTA. Using a high binding masked microtiter plate, 100 uL of ER (1 uG/well) was combined with 2 nM [³H]-17β-estradiol and various concentrations of compound. After between 5 and 15 hours at room temperature, the plates were washed with DPBS/1 mM EDTA and bound radioactivity determined by liquid scintillation counting. The IC₅₀ is defined as the concentration of compound that decreases total 17β-estradiol binding by 50%. The results obtained are described in the Table 1 below.

TABLE 1
(Selectivity of examples of this invention)

| Compound | ERβ IC₅₀ (uM) | ERα IC₅₀ (uM) |
|---|---|---|
| 17β-E₂ | 0.004 | 0.003 |
| Example 1 | 0.003 | 0.018 |
| Example 2 | 0.001 | 0.012 |
| Example 3 | 0.012 | 0.070 |
| Example 4 | 0.0012 | 0.150 |
| Example 5 | 0.017 | 1.5 |
| Example 6 | 0.005 | 0.082 |
| Example 7 | 0.003 | 0.055 |
| Example 8 | NA | NA |
| Example 9 | 0.004 | 0.129 |
| Example 10 | 0.002 | 0.159 |
| Example 11 | 0.001 | 0.042 |
| Example 12 | 0.757 | 2.79 |

The results obtained in the standard pharmacologic test procedure demonstrate that the compounds of this invention are estrogenic compounds, some with preferential affinity for ERβ, but others still possess significant binding affinity for ERα. Thus, compounds of this invention will span a range of activity based, at least partially, on their ER affinity selectivity profiles. Additionally, since each novel ER ligand complex is unique and thus, its interaction with various coregulatory proteins is unique, compounds of this invention will display different modulatory behavior depending on the cellular context they are in. For example, in some cell-types, it is possible for a compound to behave as an estrogen agonist while in other tissues, an antagonist. Compounds with such activity have sometimes been referred to as SERMs (Selective ER Modulators). Unlike many estrogens, however, many of the SERMs do not cause increases in uterine wet weight. These compounds are antiestrogenic in the uterus and can completely antagonize the trophic effects of estrogen agonists in uterine tissue. These compounds, however, may act primarily as estrogen agonists in the bone and cardiovascular systems. Due to this tissue selective nature of these compounds, they are useful in treating or preventing in a mammal, disease states or syndromes that are caused or associated with an estrogen deficiency (in certain tissues such as bone or cardiovascular) or an excess of estrogen (in the uterus or mammary glands).

Even beyond such cell-specific modulation, compounds of this invention also have the potential to behave as agonists on one ER type while behaving as antagonists on the other. For example, it has been demonstrated that compounds can be an antagonist on ERβ while being an agonist on ERα (Meyers, M. J., Sun, J., Carlson, K. E., Katzenellenbogen, B. S., Katzenellenbogen, J. A., J. Med. Chem. (1999), 42(13): 2456-2468). Such ERSAA (ER Selective Agonist Antagonist) activity provides for pharmacologically distinct estrogenic activity within this series of compounds.

Standard pharmacological test procedures are readily available to determine the activity profile of a given test compound. The following briefly summarizes several representative test procedures. Standard pharmacological test procedures for SERMs also are provided in U.S. Pat. Nos. 4,418,068 and 5,998,402, which are hereby incorporated by reference in their entirety.

Rat Uterotrophic/Antiuterotrophic Test Procedure

The estrogenic and antiestrogenic properties of the compounds were determined in an immature rat uterotrophic assay (4 days. See L. J. Black and R. L. Goode, Life Sciences, 26, 1453 (1980)). Immature Sprague-Dawley rats (female, 18 days old) were tested in groups of six. The animals were treated by daily intraperitoneal injection with 10 μG compound, 100 μG compound, 100 μG compound+1 μG 17β-estradiol to check antiestrogenicity, and 1 G 17β-estradiol, with 50% DMSO/50% saline as the injection vehicle. On day 4, the animals were sacrificed by CO₂ asphyxiation and their uteri removed and stripped of excess lipid, and any fluid was removed and the wet weight determined. A small section of one horn was submitted for histology and the remainder used to isolate total RNA in order to evaluate complement component 3 gene expression.

6-Week Ovariectomized Rat Test Procedure—Bone and Cardioprotection

Female Sprague Dawley CD rats, ovx or sham ovx, are obtained 1 day after surgery from Taconic Farm (Germantown, N.Y.) (weight range 240-275 g). They are housed 3 or 4 rats/cage in a room on a 12/12 (light/dark) schedule and provided with food (Purina® 5K96C rat chow) and water ad libitum. Treatment for all studies begin 1 day after the animals arrival and dosed 7 days per week as indicated for 6 weeks. A group of age matched sham operated rats not receiving any treatment serve as an intact, estrogen replete control group for each study.

All treatments are prepared in 1% Tween® 80 in normal saline at defined concentrations so that the treatment volume is 0.1 mL/100 g body weight. 17β-estradiol is dissolved in corn oil (20 μg/mL) and delivered subcutaneously, 0.1 mL/rat. All dosages are adjusted at three week intervals according to group mean body weight measurements.

Five weeks after the initiation of treatment and one week prior to the termination of the study, each rat is evaluated for bone mineral density (BMD). The total and trabecular density of the proximal tibia are evaluated in anesthetized rats using an XCT-960M (pQCT; Stratec Medizintechnik, Pforzheim, Germany). The measurements are performed as follows: Fifteen minutes prior to scanning, each rat is anesthetized with an intraperitoneal injection of 45 mg/kg ketamine, 8.5 mg/kg xylazine, and 1.5 mg/kg acepromazine.

The right hind limb is passed through a polycarbonate tube with a diameter of 25 mm and taped to an acrylic frame with the ankle joint at a 90° angle and the knee joint at 180°. The polycarbonate tube is affixed to a sliding platform that maintains it perpendicular to the aperture of the pQCT. The platform is adjusted so that the distal end of the femur and the proximal end of the tibia would be in the scanning field. A two dimensional scout view is run for a length of 10 mm and a line resolution of 0.2 mm. After the scout view is displayed on the monitor, the proximal end of the tibia is located. The pQCT scan is initiated 3.4 mm distal from this point. The pQCT scan is 1 mm thick, has a voxel (three dimensional pixel) size of 0.140 mm, and consists of 145 projections through the slice.

After the pQCT scan is completed, the image is displayed on the monitor. A region of interest, including the tibia but excluding the fibula, is outlined. The soft tissue is automatically removed using an iterative algorithm. The density of the remaining bone (total density) is reported in mg/cm³. The outer 55% of the bone is peeled away in a concentric spiral. The density of the remaining bone (Trabecular density) is reported in mg/cm³. One week after BMD evaluation the rats are euthanized by carbon dioxide suffocation and blood collected for cholesterol determination. The uteri are removed and the weights taken. Total cholesterol is determined using a Boehringer-Mannheim Hitachi 911 clinical analyzer (Ingelheim, Germany) using the Cholesterol/HP kit. Statitstics were compared using one-way analysis of variance with Dunnet's test.

MCF-7/ERE Antiproliferative Test Procedure

Stock solutions of test compounds (usually 0.1 M) are prepared in DMSO and then diluted 10 to 100-fold with DMSO to make working solutions of 1 or 10 mM. The DMSO stocks are stored at either 4° C. (0.1M) or −20° C. (<0.1 M). MCF-7 cells are passaged twice a week with growth medium [D-MEM/F-12 medium containing 10% (v/v) heat-inactivated fetal bovine serum, 1% (v/v) Penicillin-Streptomycin, and 2 mM glutaMax-1]. The cells are maintained in vented flasks at 37° C. inside a 5% CO₂/95% humidified air incubator. One day prior to treatment, the cells are plated with growth medium at 25,000/well into 96 well plates and incubated at 37° C. overnight.

The cells are infected for 2 hr at 37° C. with 50 μl/well of a 1:10 dilution of adenovirus 5-ERE-tk-luciferase in experimental medium [phenol red-free D-MEM/F-12 medium containing 10% (v/v) heat-inactived charcoal-stripped fetal bovine serum, 1% (v/v) Penicillin-Streptomycin, 2 mM glutaMax-1, 1 mM sodium pyruvate]. The wells then are washed once with 150 μl of experimental medium. Finally, the cells are treated for 24 hr at 37° C. in replicates of 8 wells/treatment with 150 μl/well of vehicle (≦0.1% v/v DMSO) or compound that is diluted ≧1000-fold into experimental medium.

Initial screening of test compounds is done at a single dose of 1 μM that is tested alone (agonist mode) or in combination with 0.1 nM 17β-estradiol (EC₈₀; antagonist mode). Each 96 well plate also includes a vehicle control group (0.1% v/v DMSO) and an agonist control group (either 0.1 or 1 nM 17β-estradiol). Dose-response experiments are performed in either the agonist and/or antagonist modes on active compounds in log increases from 10⁻¹⁴ to 10⁻⁵ M. From these dose-response curves, EC₅₀ and IC₅₀ values, respectively, are generated. The final well in each treatment group contains 5 μl of 3×10⁻⁵ M ICI-182,780 (10⁻⁶ M final concentration) as an ER antagonist control.

After treatment, the cells are lysed on a shaker for 15 min. with 25 μl/well of 1× cell culture lysis reagent (Promega Corporation, Madison, Wis.). The cell lysates (20 μl) are transferred to a 96 well luminometer plate, and luciferase activity is measured in a MicroLumat LB 96 P luminometer (EG & G Berthold, Wildbad, Germany) using 100 μl/well of luciferase substrate (Promega Corporation). Prior to the injection of substrate, a 1 second background measurement is made for each well. Following the injection of substrate, luciferase activity is measured for 10 seconds after a 1 second delay. The data are transferred from the luminometer to a Macintosh personal computer and analyzed using the JMP software (SAS Institute, Cary, N.C.); this program subtracts the background reading from the luciferase measurement for each well and then determines the mean and standard deviation of each treatment.

The luciferase data are transformed by logarithms, and the Huber M-estimator is used to down-weight the outlying transformed observations. The JMP software is used to analyze the transformed and weighted data for one-way ANOVA (Dunnett's test). The compound treatments are compared to the vehicle control results in the agonist mode, or the positive agonist control results (0.1 nM 17β-estradiol) in the antagonist mode. For the initial single dose experiment, if the compound treatment results are significantly different from the appropriate control (p<0.05), then the results are reported as the percent relative to the 17β-estradiol control [i.e., ((compound−vehicle control)/(17β-estradiol control−vehicle control))×100]. The JMP software also is used to determine the EC₅₀ and/or IC₅₀ values from the non-linear dose-response curves.

Inhibition of LDL Oxidation—Antioxidant Activity

Porcine aortas are obtained from an abattoir, washed, transported in chilled PBS, and aortic endothelial cells are harvested. To harvest the cells, the intercostal vessels of the aorta are tied off and one end of the aorta clamped. Fresh, sterile filtered, 0.2% collagenase (Sigma Type I) is placed in the vessel and the other end of the vessel is then clamped to form a closed system. The aorta is incubated at 37° C. for 15-20 minutes, after which the collagenase solution is collected and centrifuged for 5 minutes at 2000×g. Each pellet is suspended in 7 mL of endothelial cell culture medium consisting of phenol red free DMEM/Ham's F12 media supplemented with charcoal stripped FBS (5%), NuSerum (5%), L-glutamine (4 mM), penicillin-streptomycin (1000 U/ml, 100 μg/ml) and gentimicin (75 μg/ml), seeded in 100 mm petri dish and incubated at 37° C. in 5% CO₂. After 20 minutes, the cells are rinsed with PBS and fresh medium added, this was repeated again at 24 hours. The cells are confluent after approximately 1 week. The endothelial cells are routinely fed twice a week and, when confluent, trypsinized and seeded at a 1:7 ratio. Cell mediated oxidation of 12.5 μg/mL LDL is allowed to proceed in the presence of the compound to be evaluated (5 μM) for 4 hours at 37° C. Results are expressed as the percent inhibition of the oxidative process as measured by the TBARS (thiobarbituric acid reactive substances) method for analysis of free aldehydes (Yagi K., Biochem Med 15:212-216 (1976)).

D12 Hypothalmic Cell Test Procedure

D12 rat hypothalamic cells are subcloned from the RCF17 parental cell line and stored frozen. They are routinely grown in DMEM:F12 (1:1), glutaMAX-1 (2 mM), penicillin (100 U/ml)-streptomycin (100 mg/ml), plus 10% fetal bovine serum (FBS). The cells are plated in phenol red-free medium (DMEM:F12, glutaMAX, penicillin-streptomycin) containing 2-10% charcoal stripped FBS at a subconfluent density (1-4×10 6 cells/150 mm dish). The cells are refed 24 hr later with medium containing 2% stripped serum. To test for agonist activity, cells are treated with 10 nM 17β-estradiol or various doses of test compound (1 mM or a range from 1 pM to 1 mM). To test for antagonist activity the cells are treated with 0.1 nM 17β-estradiol in the absence or presence of varying doses (100 pM to 1 mM) of test compound. Control dishes also are treated with DMSO as a negative control. Forty-eight hours after hormone addition, the cells are lysed and a binding test procedure performed.

For each binding test procedure, 100-150 mg protein is incubated with 10 nM ³H-R5020+100-fold excess R5020 in a 150 ml volume. Triplicate reactions (three with R5020, three without R5020) are prepared in a 96 well plate. The protein extract is added first followed by ³H-R5020 or ³H-R5020+100× unlabeled R5020. The reaction is performed for 1-2 hr at room temperature. The reaction is stopped by the addition of 100 ml cold 5% charcoal (Norit SX-4, EM Science, Gibbstown, N.J.), 0.5% dextran 69K (Pharmacia, Uppsala, Sweden) in TE pH 7.4. After 5 min at room temperature, the bound and unbound ligand are separated by centrifugation (5 min, 1000 RCF, 4° C.). The supernatant solution (˜150 ml) is removed and transferred to a scintillation vial. Following the addition of scintillation fluid (Beckman Ready Protein+, Fullerton, Calif.), the samples are counted for 1 min. in a scintillation counter.

Progesterone ER in the CNS Preoptic Area

Sixty (60) day old female Sprague-Dawley rats are ovariectomized. The animals are housed in an animal care facility with a 12-hr light, 12-hr dark photoperiod and free access to tap water and rodent chow.

Ovariectomized animals are randomly divided into groups that are injected with vehicle (50% DMSO, 40% PBS, 10% ethanol vehicle), 17β-estradiol (200 ng/kg) or the compound to be tested. Additional animals are injected with the test compound 1 hr prior to injection of 17β-estradiol to evaluate the antagonistic properties of the compound. Six hr. after subcutaneous injection, animals are euthanized with a lethal dose of CO₂ and their brains collected and frozen.

Tissue collected from animals is cut on a cryostat at −16° C. and collected on Silane-coated microscope slides. The section-mounted slides then are dried on a slide warmer maintained at 42° C. and stored in desiccated slide boxes at −80° C. Prior to processing, the desiccated slide boxes are slowly warmed to room temperature (−20° C. for 12-16 hrs; 4° C. for 2 hrs; room temperature for 1 hr) to eliminate condensation formation on slides and thus, minimize tissue and RNA degradation. The dry slides are loaded into metal racks, postfixed in 4% paraformaldehyde (pH 9.0) for 5 min and processed as previously described.

A plasmid containing 815 bp fragment of the rat PR cDNA 9 (ligand binding domain) is linearized and used to generate a S 35-UTP labeled probe that is complimentary to a portion of the rat PR mRNA. Processed section-mounted slides are hybridized with 20 ml of hybridization mix containing the riboprobe (4-6×106 DPM/slide) and 50% formamide and incubated overnight in a 55° C. humidified chamber. In the morning, the slides are placed in metal racks that are immersed in 2×SSC (0.15M NaCl, 0.015M sodium citrate; pH 7.0)/10 mM DTT. All the racks are transferred to a large container and washed in 2×SSC/10 mM DTT for 15 min at room temperature with gentle agitation. The slides then are washed in RNase buffer at 37° C. for 30 min, treated with RNase A (2 mg/ml) for 30 min at 37° C., and washed for 15 min in room temperature 1×SSC. Subsequently, the slides are washed (2×30 min) in 65° C. 0.1×SSC to remove nonspecific label, then rinsed in room temperature 0.1×SSC for 15 min and dehydrated with a graded series of alcohol: ammonium acetate (70%, 95%, and 100%). Air dried slides are exposed to x-ray film for 3 days and then photographically processed. The slides from all animals are hybridized, washed, exposed and photographically processed together to eliminate differences due to interassay variation in conditions.

Rat Hot Flush—CNS Effects

Ovariectomized-female, 60 day-old Sprague-Dawley rats are obtained following surgery. The surgeries are done a minimum of 8 days prior to the first treatment. The animals are housed individually under 12 hr light/dark cycle and given standard rat chow and water ad libitum.

Two control groups are included in every study. Doses are prepared based on mg/kg mean group body weight in either 10% DMSO in sesame oil (subcutaneous (sc) studies) or in 1.0% Tween® 80 in saline (oral (po) studies). Animals are administered test compounds at doses ranging from 0.01 to 10 mg/kg mean group body weight. Vehicle and ethinyl estradiol (EE) controls (0.1 mg/kg, sc or 0.3 mg/kg, po) control groups are included in each test. When the compounds are tested for their antagonist activity, EE is coadministered at 0.1 or 0.3 mg/kg for sc or po studies, respectively. The test compounds are administered up to the day tail skin temperature is measured.

After the acclimation period of four days, the animals are treated once daily with the compound(s) of interest. There are 10 animals/treatment group. Administration of the compound is either by sc injection of 0.1 ml in the nape of the neck or po in a volume of 0.5 ml. On the 3rd day of treatment, a morphine pellet (75 mg morphine sulfate) is implanted subcutaneously. On the 5th day of treatment, one or two additional morphine pellets are implanted. On the eighth day, approximately half of the animals are injected with Ketamine (80 mg/kg, intramuscularly) and a thermocouple, connected to a MacLab Data Acquisition System (API Insturments, Milford, Mass.) is taped on the tail approximately one inch from the root of the tail. This system allowed the continuous measurement of tail skin temperature. Baseline temperature is measured for 15 min, then naloxone (1.0 mg/kg) is given sc (0.2 ml) to block the effect of morphine and tail skin temperature is measured for one hour thereafter. On the ninth day, the remaining animals are set up and analyzed similarly.

Vasomotor Function in Isolated Rat Aortic Rings

Sprage-Dawley rats (240-260 grams) are divided into 4 groups:

1. Normal non-ovariectomized (intact)
2. Ovariectomized (ovex) vehicle treated
3. Ovariectomized 17-β estradiol treated (1 mg/kg/day)
4. Ovariectomized animals treated with test compound (i.e., 1 mg/kg/day)

Animals are ovariectomized approximately 3 weeks prior to treatment. Each animal receives 1 mg/kg/day of either 17-β estradiol sulfate or test compound suspended in distilled, deionized water with 1% Tween® 80 by gastric gavage. Vehicle treated animals received an appropriate volume of the vehicle used in the drug treated groups.

Animals are euthanized by CO₂ inhalation and exsanguination. Their thoracic aortas are removed rapidly and placed in 37° C. physiological solution with the following composition (mM): NaCl (54.7), KCl (5.0), NaHCO₃ (25.0), MgCl₂ 2H₂O (2.5), D-glucose (11.8) and CaCl₂ (0.2) gassed with CO₂—O₂, 95%/5% for a final pH of 7.4. The advantitia is removed from the outer surface and the vessel is cut into 2-3 mm wide rings. The rings are suspended in a 10 mL tissue bath with one end attached to the bottom of the bath and the other to a force transducer. A resting tension of 1 gram is placed on the rings. The rings are equilibrated for 1 h, and signals are acquired and analyzed.

After equilibration, the rings are exposed to increasing concentrations of phenylephrine (10⁻⁸ to 10⁻⁴ M) and the tension recorded. The baths then are rinsed 3 times with fresh buffer. After washout, 200 mM L-NAME is added to the tissue bath and equilibrated for 30 minutes. The phenylephrine concentration response curve is then repeated.

Eight Arm Radial Arm Maze—Cognition Enhancement

Male Sprague-Dawley, CD rats (Charles River, Kingston, N.Y.) weighing 200-250 g on arrival are used. For one week, the rats are housed, six per cage, with standard laboratory chow and water available ad libitum. Housing is in a colony room maintained at 22° C. that has a 12 hour light/dark cycle with lights on at 6:00 AM. Following habituation to the facility, animals are individually housed and maintained at 85% of free-feeding weight. Once stable weights are attained, the rats are acclimated to the 8-arm radial maze.

The structure of the maze is an adaptation from that of Peele and Baron (Pharmacology, Biochemistry, and Behavior, 29:143-150, (1988)). The maze is elevated to a height of 75.5 cm and composed of a circular area surrounded by 8 arms radiating away from the center, equidistant from one another. Each arm is 58 cm long×13 cm high. A clear plexiglass cylinder is lowered to enclose the animal in the center portion of the maze prior to the start of each session. Each arm of the maze is equipped with 3 sets of photocells interfaced to a data acquisition unit, which in turn is interfaced to a computer. The photocells are used to track the movement of the rat in the maze. Pellet feeders located above food cups at the end of each arm, dispensed two 45 mg chocolate pellets when the outer photocell of the arm is activated for the first time in a given session. The maze is located in a testing room with black and white geometric posters on each wall to serve as visual cues. During all training and testing procedures, white noise is audible (˜70 db).

The training procedure consists of five phases, each with daily sessions lasting 5 or 10 minutes. A 10 second delay is imposed between the time the rat is placed in the center portion of the maze and when the cylinder is raised to begin the session. During Phase 1, food-restricted pairs of rats are placed on the maze for 10 minutes with 45 mg chocolate food pellets scattered throughout the 8 arms of the maze. During Phase II, each rat is placed individually on the maze for a 10 minute period, with pellets scattered from the middle photocell to the food cup of each arm. During Phase III, each rat is placed on the maze for a 10 minute period, with food pellets located only in and around the food cups in each arm. In Phase IV, each rat is allowed 10 minutes to collect two pellets from each arm. Re-entry into an arm is considered an error. Rats are trained daily in this manner until they achieved criterion performance with less than or equal to 2 total errors on three consecutive days of training. Total habituation and training time is approximately 3 weeks.

Test compound is prepared in phosphate buffered saline and administered in a volume of 1 ml/kg. Scopolamine HBr (0.3 mg/kg s.c.) served as the impairing agent, producing an increase in error rate (loss of memory). Test compound is given intraperitoneally simultaneously with scopolamine, 30 minutes prior to the first maze exposure on any given test day.

To assess the test compound, an 8×8 balanced latin square for repeated measures is designed, in order to achieve a high experimental efficiency with the least amount of animals. Eight experimental sessions, two per week, are conducted with the 8 treatments (vehicle, scopolamine, 3 doses of test compound in combination with scopolamine), randomized within each session. Each treatment followed every other treatment the same number of times. Therefore, the residual effect of every treatment could be estimated and removed from the direct treatment effect. Following ANOVA, multiple comparisons are performed using Dunnett's two-sided test on adjusted means.

Animals that did not make four correct choices within 5 minutes during the first exposure, or that had not made a total of 8 choices by the end of the second exposure, are considered to have “timed-out” for that session. Any animal that “timed-out” following administration of more than one dose of the test compound is excluded from the analysis.

Neuroprotection

Inhibition of Time-Dependent Death of Cells in Primary Cortical Neuron Cultures

Primary cortical neurons were produced from rat brains that were 0-1 day old using a variation of methods described by Monyer et al. Brain Research ((1989), 483:347-354). Dispersed brain tissue was grown in DMEM/10% PDHS (pregnant donor horse serum) for three days and then treated with cytosine arabinoside (ARC) for two days to remove contaminating glial cells. On day 5, the ARC media was removed and replaced with DMEM/10% PDHS. The neuronal cells were cultured for a further 4-7 days before use.

Control primary neuronal cultures show progressive cell death between days 12 and 18 in culture. Twelve cultures were evaluated on days 12 and 16 for levels of the enzyme lactate dehydrogenase (LD), after adding on day 9, test compound to 6 cultures maintained in DMEM and 10% PDHS while maintaining the remaining cultures as controls. LD was assayed using a variation of the method by Wroblewski et al. Proc. Soc. Exp. Biol. Med. ((1955) 90:210-213). LD is a cytosolic enzyme that is commonly used in both clinical and basic research to determine tissue viability. An increase in media LD is directly related to cell death.

Neuroprotection against Cytotoxicity Induced by Hypoglycemia

C6 glioma cells obtained from American Type Culture Collection (ATCC) were plated in RPMI media with FBS at a concentration of 1×10⁶ cells/ml in FALCON™ 25 cm² tissue culture flasks. Four hours prior to the onset of hypoglycemia, the maintenance media was discarded, monolayers were washed twice in the appropriate media and then incubated for four hours at 37° C. in either serum free or serum free plus test compound. Kreb's Ringer Phosphate buffer was used to wash the monolayers twice before the addition of appropriate glucose treatment. RPMI medium contains 2 mg glucose/ml. Flasks were divided into groups of six, each receiving 100% glucose (2 mg/ml), 80% glucose (1.6 mg/ml), 60% glucose (1.2 mg/ml) or 0% glucose (buffer) or supplemented with test compound. All flasks were incubated for 20 hours and then evaluated for total, live, and dead cell number utilizing trypan blue.

Neuroprotection against Excitotoxic Amino Acids

Five culture dishes containing SK—N—SH neuroblastoma cells were treated with test compound and 5 culture dishes were treated with RPMI media. Four hours later, all cell were treated with NMDA (500 μM) for 5 minutes. Total live cells and dead cells were then determined.

Neuroprotection against Oxygen-Glucose Deprivation

Analysis of Pyknotic Nuclei to Measure Apoptosis

Cortical neurons are prepared from E18 rat fetus and plated in 8-well chamber slides precoated with poly-D-lysine (10 ng/ml) and serum at a density of 100,000 cells/well. Cells are plated in high glucose DMEM containing 10% FCS and kept in the incubator at 37° C. with 10% CO₂/90% air. On the next day, serum is removed by replacing culture media with high glucose DMEM containing B27 supplement and cells are kept in the incubator without further media change until the day of experiment. On day 6, slides are divided into two groups; a control group and and Oxygen-Glucose Deprived (OGD) group. Cells in the control group receive DMEM with glucose and custom B27 (without antioxidants). Cells in the OGD group receive no-glucose DMEM with custom B27, which has been degassed under vacuum for 15 min. Cells are flushed with 90% N₂/10% CO₂ for 10 min in an airtight chamber and incubated at 37° C. for 6 hrs. After 6 hrs, both control and OGD cells are subject to replacement of media containing either vehicle (DMSO) or test compound in glucose-containing DMEM with custom B27. Cells are returned to a normoxic incubator at 37° C. After 24 hrs, cells are fixed in 4% PFA for 10 min at 4° C. and stained with To-Pro (fluorescent nuclear binding dye). Apoptosis is assessed using a Laser Scanning Cytometer by measuring pyknotic nuclei.

Measurement of Lactate Dehydrogenase (LDH) Release as an Indication of Cell Death

Cortical neurons are prepared from E18 rat fetus and plated in 48-well culture plates precoated with poly-D-lysine (10 ng/ml) and serum at a density of 150,000 cells/well. Cells are plated in high glucose DMEM containing 10% FCS and kept in the incubator at 37° C. with 10% CO₂/90% air. On the next day, serum is removed by replacing culture media with high glucose DMEM containing B27 supplement. On day 6, cells are divided into two groups: a control group and an OGD group. Cells in the control group receive DMEM with glucose and custom B27 (without antioxidants). Cells in the OGD group receive no-glucose DMEM with custom B27, which has been degassed under vacuum for 15 min. Cells are flushed with 90% N₂/10% CO₂ for 10 min in an airtight chamber and incubated at 37° C. for 6 hrs. After 6 hrs, both control and OGD cells are subject to replacement of media containing either vehicle (DMSO) or test compound in glucose-containing DMEM with custom B27. Cells are returned to normoxic incubator at 37° C. After 24 hrs, cell death is assessed by measuring cellular release of LDH (lactate dehydrogenase) into the culture medium. For LDH assay, an aliquot of 50 μl culture medium is transferred into the 96 well plate. After the addition of 140 μl 0.1M potassium phosphate buffer (pH 7.5) and 100 μl 0.2 mg/ml NADH, the plate is allowed to sit in the dark at room temperature for 20 min. The reaction is initiated by the addition of 10 μl of sodium pyruvate. The plate is read immediately at 340 nM in a Thermomaxe plate reader (Molecular Devices, Sunnyvale, Calif.). The absorbance, an index of NADH concentration, is recorded every 6 seconds for 5 minutes and the slope indicating the rate of NADH disappearance is used to calculate LDH activity.
LDH Activity(U/ml)=(A/min) (TCF)(20)(0.0833)/(0.78)
where:
   0.0833=proportionality constant
   0.78=instrument light path length (cm)
      HLA Rat Test Procedure—Crohn's Disease and Inflammatory Bowel Disorders

Male HLA-B27 rats are obtained from Taconic Farm (Germantown, N.Y.) and provided unrestricted access to food (PMI Lab Diets 5001) and water. At the start of the study, rats are 22-26 weeks old.

Rats are dosed subcutaneously once per day for seven days with one of the formulations listed below. There are five rats in each group and the last dose is administered two hours before euthanasia.

Formulations:
vehicle (50% DMSO/50% Dulbecco's PBS)
17α-ethinyl-17β-estradiol (10 μg/kg)
test compound

Stool quality is observed daily and graded according to the following scale: Diarrhea=3; soft stool=2; normal stool=1. At the end of the test procedure, serum is collected and stored at −70° C. A section of colon is prepared for histological analysis and an additional segment is analyzed for myeloperoxidase activity.

The following method is used to measure myeloperoxidase activity. Colon tissue is harvested and flash frozen in liquid nitrogen. A representative sample of the entire colon is used to ensure consistency between samples. The tissue is stored at −80° C. until use. Next, the tissue is weighed (approximately 500 mg) and homogenized in 1:15 w/v of 5 mM H₂ KPO₄ (pH 6) washing buffer. The tissue is spun down at 20,000×g in a Sorvall® RC 5B centrifuge for 45 minutes at 2-8° C. Supernatant is then discarded. Tissue is resuspended and homogenized in 2.5 ml (1:5 w/v) of 50 mM H₂ KPO₄ with 10 mM EDTA and 0.5% Hex Ammonium Bromide to help solubilize the intracellular myeloperoxidase (MPO). Tissue is frozen in liquid nitrogen, thawed in a 37° C.-water bath and sonicated for 15 seconds to ensure membrane lysis. This procedure is repeated 3 times. Samples then are kept on ice for 20 minutes and centrifuged at 12,000×g for 15 minutes at 2-8° C. The supernatant is analyzed following these steps.

The test mixture is prepared by adding 2.9 ml of 50 mM H₂ KPO₄ with 0.167 O-Dianisidine/ml with 0.0005% H₂O₂ into a reaction tube. When hydrogen peroxide is degraded, O-Dianisidine is oxidized and absorbs at 460 nm in a concentration dependent manner. The mixture is heated to 25° C. One hundred (100) μL of the tissue supernatant is added to the reaction tube, incubated for one minute at 25° C., then 1 ml is transferred to a disposable plastic cuvette. Optical density (OD) is measured every 2 minutes of reaction time at 460 nm against a blank containing 2.9 ml of the reaction mixture and 100 μl of the 0.5% ammonium bromide solution.

Enzyme activity units are quantified by comparison of absorbence at 460 nm to a standard curve prepared with purified human MPO, 31.1 Units/Vial. The MPO is reconstituted and serially diluted using 50 mM H₂ KPO₄ with 10 mM EDTA and 0.5% Hex Ammonium Bromide to four known concentrations. Sample absorbencies are compared against this curve to determine activity.

Histological analysis is performed as follows. Colonic tissue is immersed in 10% neutral buffered formalin. Each specimen of colon is separated into four samples for evaluation. The formalin-fixed tissues are processed in a vacuum infiltration processor for paraffin embedding. The samples are sectioned at 5 μm and then stained with hematoxylin and eosin (H&E) for blinded histologic evaluations using a scale modified after Boughton-Smith (Boughton-Smith, N. K., Wallace, J. L., Morris, G. P., Whittle, B. J., Br. J. Pharmacol. ((1988), 94: 65-72). After the scores are completed the samples are unblinded, and data are tabulated and analyzed by ANOVA linear modeling with multiple mean comparisons.

It is intended that each of the patents, applications, and printed publications, including books, mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A compound of formula I 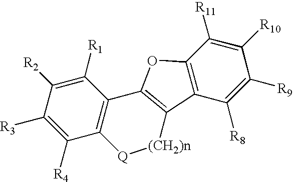
wherein:
Q has the structure II, III or IV: 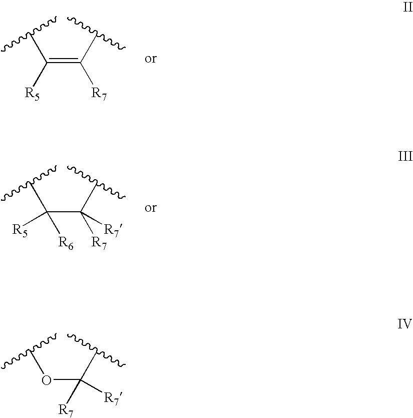
R₁, R₄, R₅, R₆, R₇, R_{7′}, R₈ and R₁₁ are each independently selected from the group consisting of hydrogen, C₁-C₆ alkyl, —OR₂₀, halogen, —CF₃, —CF₂CF₃, —CH₂CF₃, —SR₂₀, NR₂OR₂₁, —CN, —CH₂CN, —CH₂CH₂CN, —CH═CHCN, —NO₂, —CH₂NO₂, —CH₂CH₂NO₂, —CH═CHNO₂ and —COR₂₀;
n=0 or 1;
each R₂₀ and R₂₁ is independently selected from the group consisting of hydrogen, C₁-C₆ alkyl, —CF₃, benzyl, —CO₂(C₁-C₆ alkyl) and —CO(C₁-C₆ alkyl);
provided that:
a) one of R₂ or R₃ must be —OR₂₀;
b) one of R₉ or R₁₀ must be —OR₂₀;
c) when R₂ is —OR₂₀, then R₁ and R₃ are independently selected from the group consisting of hydrogen, halogen, C₁-C₆ alkyl, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
d) when R₃ is —OR₂₀, then R₂ and R₄ are independently selected from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
e) when R₉ is —OR₂₀, then R₈ and R₁₀ are independently selected from the group consisting of hydrogen, C₁-C₆ alkyl, halogen, —CF₃, —CF₂CF₃, —CH₂CF₃′—SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
f) when R₁₀ is —OR₂₀, then R₉ and R₁₁ are independently selected from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀; and
g) when Q has the structure IV, and R₇, R_{7′}, R₈, R₉, R₁₁ are each H, and n=0, then R₁₀ is not OR₂₀;
or a pharmaceutically acceptable salt thereof.

2. The compound of claim 1 wherein Q has the structure II.

3. The compound of claim 2 wherein R₃ and R₉ are each independently OR₂₀.

4. The compound of claim 2 wherein R₃ and R₁₀ are each independently OR₂₀.

5. The compound of claim 2 wherein R₂ and R₉ are each independently OR₂₀.

6. The compound of claim 2 wherein R₂ and R₁₀ are each independently OR₂₀.

7. The compound of claim 3 wherein R₁, R₂, R₄, R₈ and R₁₀ are each independently selected from the group consisting of hydrogen and halogen; and R₁₁ is selected from the group consisting of CN, halogen, methoxy, CH₂CN, NO₂, and C₁-C₆ alkyl.

8. The compound of claim 7 wherein n is 0.

9. The compound of claim 7 wherein n is 1.

10. The compound of claim 1 wherein Q has the structure III.

11. The compound of claim 10 wherein R₃ and R₉ are each independently OR₂₀.

12. The compound of claim 10 wherein R₃ and R₁₀ are each independently OR₂₀.

13. The compound of claim 10 wherein R₂ and R₉ are each independently OR₂₀.

14. The compound of claim 10 wherein R₂ and R₁₀ are each independently OR₂₀.

15. The compound of claim 11 wherein R₂, R₄, R₈ and R₁₀ are each independently selected from the group consisting of hydrogen and halogen; and R₁₁ is selected from the group consisting of CN, halogen, methoxy, CH₂CN, NO₂, and C₁-C₆ alkyl.

16. The compound of claim 15 wherein n is 0.

17. The compound of claim 15 wherein n is 1.

18. The compound of claim 1 wherein Q has the structure IV.

19. The compound of claim 18 wherein R₃ and R₉ are each independently OR₂₀.

20. The compound of claim 18 wherein R₃ and R₁₀ are each independently OR₂₀.

21. The compound of claim 18 wherein R₂ and R₉ are each independently OR₂₀.

22. The compound of claim 18 wherein R₂ and R₁₀ are each independently OR₂₀.

23. The compound of claim 19 wherein R₂, R₄, R₈ and R₁₀ are each independently selected from the group consisting of hydrogen and halogen; and R₁₁ is selected from the group consisting of CN, halogen, methoxy, CH₂CN, NO₂, and C₁-C₆ alkyl.

24. The compound of claim 23 wherein n is 0.

25. The compound of claim 23 wherein n is 1.

26. The compound of claim 1 having the structure 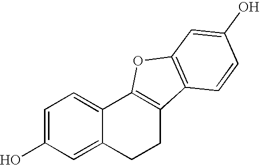
or a pharmaceutically acceptable salt thereof.

27. The compound of claim 1 having the structure 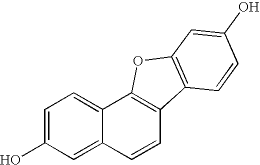
or a pharmaceutically acceptable salt thereof.

28. The compound of claim 1 having the structure 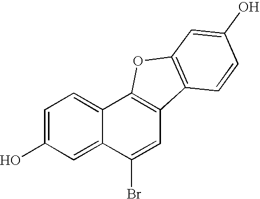
or a pharmaceutically acceptable salt thereof.

29. The compound of claim 1 having the structure 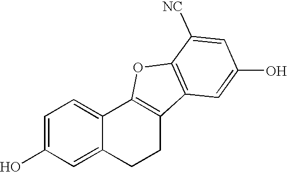
or a pharmaceutically acceptable salt thereof.

30. The compound of claim 1 having the structure 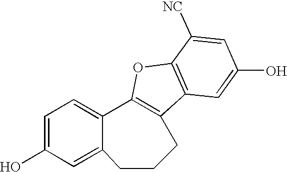
or a pharmaceutically acceptable salt thereof.

31. The compound of claim 1 having the structure 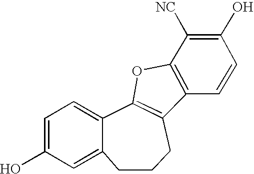
or a pharmaceutically acceptable salt thereof.

32. The compound of claim 1 having the structure 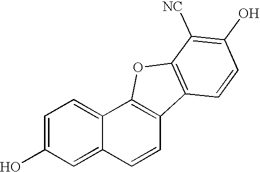
or a pharmaceutically acceptable salt thereof.

33. The compound of claim 1 having the structure 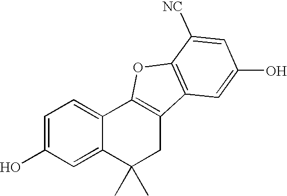
or a pharmaceutically acceptable salt thereof.

34. The compound of claim 1 having the structure 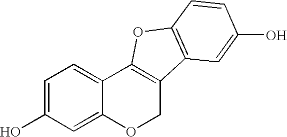
or a pharmaceutically acceptable salt thereof.

35. The compound of claim 1 having the structure 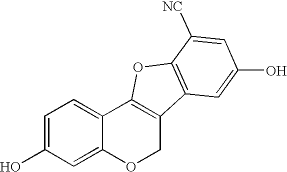
or a pharmaceutically acceptable salt thereof.

36. The compound of claim 1 having the structure 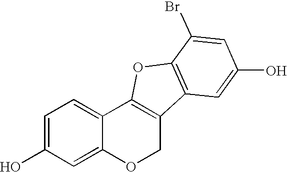
or a pharmaceutically acceptable salt thereof.

37. The compound of claim 1 having the structure 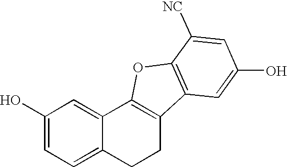
or a pharmaceutically acceptable salt thereof.

38. The compound of claim 1 having the structure 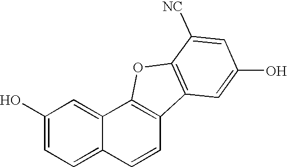
or a pharmaceutically acceptable salt thereof.

39. A method of treating or inhibiting osteoporosis or inhibiting bone demineralization in a mammal, which comprises providing to said mammal an effective amount of a compound of claim 1.

40. A method of treating or inhibiting inflammatory bowel disease, Crohn's disease, ulcerative proctitis, or colitis in a mammal, which comprises providing to said mammal an effective amount of a compound of claim 1.

41. A method of treating or inhibiting prostatic hypertrophy, uterine leiomyomas, breast cancer, polycystic ovary syndrome, endometrial polyps, benign breast disease, adenomyosis, ovarian cancer, melanoma, prostate cancer, colon cancer, glioma or astioblastomia in a mammal, which comprises providing to said mammal an effective amount of a compound of claim 1.

42. A method of lowering cholesterol, triglycerides, Lp(a), or LDL levels, or of inhibiting or treating hypercholesteremia, hyperlipidemia, cardiovascular disease, artheroclerosis, peripheral vascular disease, restenosis, or vasospasm, or inhibiting vascular damage in a mammal, which comprises providing to said mammal an effective amount of a compound of claim 1.

43. A method of providing cognition enhancement or neuroprotection, or treating or inhibiting senile dementias, Alzheimer's disease, cognitive decline, stroke, anxiety, or neurodegenrative disorders in a mammal, which comprises providing to said mammal an effective amount of a compound of claim 1.

44. A method of treating or inhibiting free radical induced disease states in a mammal, which comprises providing to said mammal an effective amount of a compound of claim 1.

45. A method of treating or inhibiting vaginal or vulvar atrophy, atrophic vaginitis, vaginal dryness, pruritus, dyspareunia, dysuria, frequent urination, urinary incontinence, or urinary tract infections in a mammal which comprises providing to said mammal an effective amount of a compound of claim 1.

46. A method of treating or inhibiting vasomotor symptoms in a mammal, which comprises providing to said mammal an effective amount of a compound of claim 1.

47. A method of contraception in a mammal, which comprises providing to said mammal an effective amount of a compound of claim 1.

48. A method of treating or inhibiting rheumatoid arthritis, osteoarthritis, or spondyloarthropathies in a mammal, which comprises providing to said mammal an effective amount of a compound of claim 1.

49. A method of treating or inhibiting joint damage secondary to arthroscopic or surgical procedures in a mammal, which comprises providing to said mammal an effective amount of a compound of claim 1.

50. A method of treating or inhibiting fertility in a mammal, which comprises providing to said mammal an effective amount of a compound of claim 1.

51. A method of treating or inhibiting ischemia, reperfusion injury, asthma, pleurisy, multiple sclerosis, systemic lupus erythematosis, uveitis, sepsis, hemorrhagic shock, or type II diabetes in a mammal, which comprises providing to said mammal an effective amount of a compound of claim 1.

52. A pharmaceutical composition comprising a compound of claim 1 or combinations thereof, and one or more pharmaceutically acceptable carriers.

53. A pharmaceutical composition comprising one or more of the following compounds:
a) 5,6-Dihydro-benzo[b]naphtho[2,1-d]furan-3,9-diol;
b) Benzo[b]naphtho[2,1-d]furan-3,9-diol;
c) 5-Bromo-benzo[b]naphtho[2,1-d]furan-3,9-diol;
d) 3,8-Dihydroxy-5,6-dihydro-benzo[b]naphtho[2,1-d]furan-10-carbonitrile;
e) 3,9-Dihydroxy-6,7-dihydro-5H-12-oxa-dibenzo[a,e]azulen-11-carbonitrile;
f) 3,9-Dihydroxy-5,6-dihydro-benzo[b]naphtho[2,1-d]furan-10-carbonitrile;
g) 3,9-Dihydroxy-benzo[b]naphtho[2,1-d]furan-10-carbonitrile;
h) 3,8-Dihydroxy-5,5-dimethyl-5,6-dihydro-benzo[b]naphtho[2,1-d]furan-10-carbonitrile;
i) 6H-Benzo[4,5]furo[3,2-c]chromen-3,8-diol;
j) 3,8-Dihydroxy-6H-Benzo[4,5]furo[3,2-c]chromene-10-carbonitrile;
k) 10-Bromo-6H-benzo[4,5]furo[3,2-c]chromene-3,8-diol;
l) 2,9-Dihydroxy-5,6-dihydro-benzo[b]naphtho[2,1-d]furan-10-benzonitrile;
m) 2,9-Dihydroxy-benzo[b]naphtho[2,1-d]furan-10-carbonitrile;
or a pharmaceutically acceptable salt thereof, and one or more pharmaceutically acceptable carriers.

54. A process for the preparation of a compound of claim 1 comprising the steps of:
a) coupling a compound of formula V 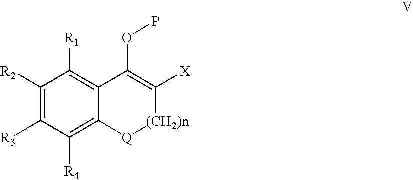
wherein X is Cl, Br, or I; and
P is a protecting group;
with a compound of formula VI 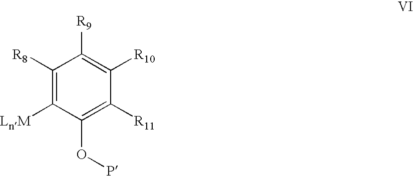
wherein
M is a metal; and
L is a ligand;
P′ is H or a protecting group; and
n′ is an integer from 0 to 5, to form a compound of formula VII; and 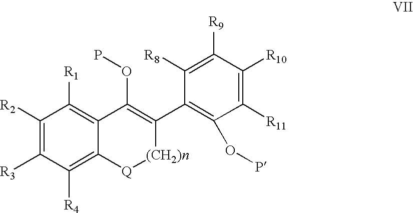
b) removing the groups P and P′ and cyclizing the resulting deprotected compound to form the compound of formula I 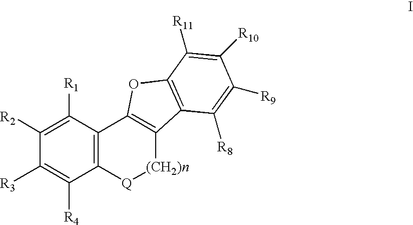
wherein R₁, R₂, R₃, R₄, R₅, R₆, Q, n, R_{7′}, R₇, R₈, R₉, R₁₀, R₁₁ are as defined in claim 1.

55. The process of claim 54 wherein
P is Si(R′)₃; COC₁-C₆ alkyl, COOC₁-C₆ alkyl, CObenzyl, CO₂benzyl, C₁-C₆ alkyl; and each R′ is independently C₁-C₆ alkyl or phenyl; and
P′ is H, Si(R′)₃; COC—C₆ alkyl, COOC₁-C₆ alkyl, CObenzyl, C₁-C₆ alkyl;
wherein each R′ is independently selected from a group consisting of C₁-C₆ alkyl or phenyl.

56. The process of claim 55 wherein
P is COC₁-C₆ alkyl, COOC₁-C₆ alkyl, CObenzyl, CO₂benzyl; and
P′ is C₁-C₆ alkyl; and
M is B; and
L is (OH) or (OC₁-C₆ alkyl); and n′ is 2; or
M is Sn; and
L is (C₁-C₆ alkyl); and n′ is 3.

57. The process of claim 56 wherein P in step b) is removed with an organic or inorganic hydroxide and P′ in step b) is removed with boron tribromide, hydroiodic acid, pyridine hydrochloride or pyridine hydrobromide.

58. The process of claim 57 wherein the cyclization occurs during the removal of P′.

59. A compound prepared by the process of any of claims 54-58.

60. A process for preparing a compound of formula I according to claim 1, which comprises cyclizing a compound of formula: 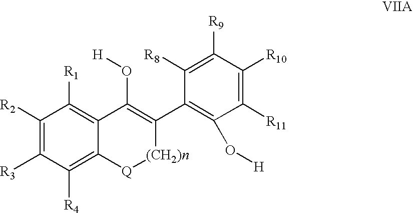
wherein n, R₁-R₄ and R₈-R₁₁ are as defined in claim 1 to form the compound of Formula I; and
optionally isolating said compound of Formula I as a pharmaceutically acceptable salt thereof.
